(19) United States
(12) Patent Application Publication
(10) Pub. No.: US 2003/0036641 A1
(43) Pub. Date: Feb. 20, 2003

(54) Methods for homology-driven reassembly of nucleic acid sequences

(76) Inventors: Hal S. Padgett, Vacaville, CA (US); Wayne P. Fitzmaurice, Vacaville, CA (US); John A. Lindbo, Vacaville, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/775,049

(22) Filed: Jan. 31, 2001

(51) Int. Cl. C07H 21/04; C12P 19/34

(52) U.S. Cl. 536/23.1; 435/91.2

(57) ABSTRACT

The invention provides methods of forcing recombination between polynucleotides. The methods can include the steps of, (a) generating a single strand of a first polynucleotide; (b) generating a single strand of a second polynucleotide, wherein the second polynucleotide is partially complementary to the first polynucleotide; (c) fragmenting the single strand of the first polynucleotide to generate single stranded first polynucleotide fragments; (d) fragmenting the single strand of the second polynucleotide to generate single stranded second polynucleotide fragments; (e) annealing the single stranded first polynucleotide fragments with the single stranded second polynucleotide fragments; and (f) extending the annealed polynucleotide fragments.

### BACKGROUND OF THE INVENTION

### FIELD OF THE INVENTION

The invention relates generally to molecular biology and more specifically to methods of generating populations of related nucleic acid molecules.

An exceedingly large number of possibilities exist for purposeful and random combinations of amino acids within a protein to produce useful mutant proteins and their corresponding biological molecules encoding for the mutant proteins, i.e., DNA, RNA, etc. Accordingly, there is a need to produce and screen a wide variety of such mutant proteins for variouse uses, particularly widely varying random proteins.

The complexity of an active sequence of a biological macromolecule, e.g., proteins, DNA etc., has been called its information content (“IC”)(Stormo, G. D. (1991) Methods Enzymol. 208:458-468; Schneider, T. D. et al., (1986) J. Mol. Biol. 188:415-431; Reidhaar-Olson, J. F and Sauer, R. T. (1988) Science 241:53-57; Stemmer, W. P. C. et al., (1993) Biotechniques 14:256-265; Yockey, H. P. (1977) J. Theor. Biol. 67:345-376), which has been defined as the resistance of the active protein to amino acid sequence variation (calculated from the minimum number of invariable amino acids (bits)) required to describe a family of related sequences with the same function (Yockey, H. P. (1977) J. Theor. Biol. 67:345-376; Yockey, H. P. (1974) J. Theor. Biol. 46:369-380). Proteins that are more sensitive to random mutagenesis have a high information content.

Molecular biology developments such as molecular libraries have allowed the identification of quite a large number of variable bases, and even provide ways to select functional sequences from random libraries. In such libraries, most residues can be varied (although typically not all at the same time) depending on compensating changes in the context. Thus, while a 100 amino acid protein can contain only 2,000 different mutations, 20¹⁰⁰ combinations of mutations are possible.

Information density is the Information Content per unit length of a sequence. Active sites of enzymes tend to have a high information density. By contrast, flexible linkers of information in enzymes have a low information density (Stemmer, W. P. C. et al., (1993) Biotechniques 14:256-265).

Current methods in widespread use for creating mutant proteins in a library format are error-prone polymerase chain reactions (Cadwell, R. C. and Joyce, G. F. (1992) PCR Methods and Applications 2:28-33; Gram, H. et al., (1992) Proc. Natl. Acad. Sci. USA 89:3576-3580) and cassette mutagenesis (Stemmer, W. P. C. et al., (1993) Biotechniques 14:256-265; Arkin, A. P. and Youvan, D. C. (1992) Proc. Natl. Acad. Sci. USA 89:7811-7815; Oliphant, A. R. et al., (1986) Gene 44:177-183; Hermes, J. D. et al., (1990) Proc. Natl. Acad. Sci. USA 87:696-700; Delagrave et al. (1993) Protein Engineering 6: 327-331; Delgrave et al. (1993) Bio/Technology 11: 1548-1552; Goldman, ER and Youvan DC (1992) Bio/Technology 10:1557-1561) in which the specific region to be optimized is replaced with a synthetically mutagenized oligonucleotide. In both cases, a cloud of mutant sites (Kauffman, S. A. (1993) “The origins of order”. Oxford University Press, New York) is generated around certain sites in the original sequence.

Error-prone PCR uses low-fidelity polymerization conditions to introduce a low level of point mutations randomly over a long sequence. In a mixture of fragments of unknown sequence, error-prone PCR can be used to mutagenize the mixture. The published error-prone PCR protocols suffer from a low processivity of the polymerase. Therefore, the protocol is unable to result in the random mutagenesis of an average-sized gene. This inability limits the practical application of error-prone PCR. Some computer simulations have suggested that point mutagenesis alone may often be too gradual to allow the large-scale block changes that are required for continued and dramatic sequence evolution. Further, the published error-prone PCR protocols do not allow for amplification of DNA fragments greater than 0.5 to 1.0 kb, limiting their practical application. In addition, repeated cycles of error-prone PCR can lead to an accumulation of neutral mutations with undesired results, such as affecting a protein's immunogenicity but not its binding affinity.

In oligonucleotide-directed mutagenesis, a short sequence is replaced with a synthetically mutagenized oligonucleotide. This approach does not generate combinations of distant mutations and is thus not combinatorial. The limited library size relative to the vast sequence length means that many rounds of selection are unavoidable for protein optimization. Mutagenesis with synthetic oligonucleotides requires sequencing of individual clones after each selection round followed by grouping them into families, arbitrarily choosing a single family, and reducing it to a consensus motif. Such a motif is resynthesized and reinserted into a single gene followed by additional selection. This step process constitutes a statistical bottleneck, is labor intensive, and is not practical for many rounds of mutagenesis.

Error-prone PCR and oligonucleotide-directed mutagenesis are thus useful for single cycles of sequence fine tuning, but rapidly become too limiting when they are applied for multiple cycles.

Another serious limitation of error-prone PCR is that the rate of down-mutations grows with the information content of the sequence. As the information content, library size, and mutagenesis rate increase, the balance of down-mutations to up-mutations will statistically prevent the selection of further improvements (statistical ceiling).

In cassette mutagenesis, a sequence block of a single template is typically replaced by a (partially) randomized sequence. Therefore, the maximum information content that can be obtained is statistically limited by the number of random sequences (i.e., library size). This eliminates other sequence families which are not currently best, but which may have greater long term potential.

Also, mutagenesis with synthetic oligonucleotides requires sequencing of individual clones after each selection round (Arkin, A. P. and Youvan, D. C. (1992) Proc. Natl. Acad. Sci. USA 89:7811-7815). Thus, such an approach is tedious and impractical for many rounds of mutagenesis.

Thus, error-prone PCR and cassette mutagenesis are best suited, and have been widely used, for fine-tuning areas of comparatively low information content. One apparent exception is the selection of an RNA ligase ribozyme from a random library using many rounds of amplification by error-prone PCR and selection (Bartel, D. P., and Szostak, J. W. (1993) Science 261:1411-1418).

It is becoming increasingly clear that the tools for the design of recombinant linear biological sequences such as protein, RNA and DNA are not as powerful as the tools nature has developed. Finding better and better mutants depends on searching more and more sequences within larger and larger libraries, and requiring increased numbers of cycles of mutagenic amplification and selection. However as discussed above, the existing mutagenesis methods that are in widespread use have distinct limitations when used for repeated cycles.

In nature, the evolution of most organisms occurs by natural selection and sexual reproduction. Sexual reproduction ensures mixing and combining of the genes in the offspring of the selected individuals. During meiosis, homologous chromosomes from the parents line up with one another and cross-over part way along their length, thus randomly swapping genetic material. Such swapping or shuffling of the DNA allows organisms to evolve more rapidly (Holland, J. H. (1992) Sci. Am. July, 66-72; Holland, J. H. (1992) “Adaptation in natural and artificial systems”. Second edition, MIT Press, Cambridge).

In sexual recombination, because the inserted sequences were of proven usefulness in a homologous environment, the inserted sequences are likely to still have substantial information content once they are inserted into the new sequence.

Marton et al. ((1991) Nucleic Acids Res. 19:2423) describes the use of PCR in vitro to monitor recombination in a plasmid having directly repeated sequences. Marton et al. describes that recombination will occur during PCR as a result of breaking or nicking of the DNA. This will give rise to recombinant molecules. Meyerhans et al. ((1990) Nucleic Acids Res. 18:1687-1691) also describe the existence of DNA recombination during in vitro PCR.

The term Applied Molecular Evolution (“AME”) means the application of an evolutionary design algorithm to a specific, useful goal. While many different library formats for AME have been reported for polynucleotides (Joyce, G. F. (1992) Scientific American, 267:6, 90-97; Cadwell, R. C. and Joyce, G. F. (1992) PCR Methods and Applications 2:28-33; Bartel, D. P., and Szostak, J. W. (1993) Science 261:1411-1418; Bock, L. C. et al., (1992) Nature 355:564-566) peptides and proteins (peptide epitopes on phage: Scott, J. K. and Smith, G. P. (1990) Science 249:386-390; Cwirla, S. E. et al. (1990) Proc. Natl. Acad. Sci. USA 40 87:6378-6382; McCafferty, J. et al. (1990) Nature 348:552-554) (peptides on laci: Cull, M. G. et al., (1992) Proc. Natl. Acad. Sci. USA 45 89:1865-1869) and polysomes, none of these formats have provided for recombination by random cross-overs to deliberately create a combinatorial library.

Theoretically, there are 2,000 different single mutants of a 100 amino acid protein. However, a protein of 100 amino acids has 20¹⁰⁰ possible combinations of mutations, a number which is too large to exhaustively explore by conventional methods.

An in vivo site specific recombination system has been used to combine light chain antibody genes with heavy chain antibody genes for expression in a phage system (Nissim et al. (1994) EMBO J. 13: 692-698; Winter et al. (1994) Ann. Rev. Immunol. 12: 433-55). However, this system relies on specific sites of recombination and is limited accordingly. Simultaneous mutagenesis of antibody CDR regions in single chain antibodies (scFv) by overlapping extension and PCR have been reported (Hayashi et al. (1994) Biotechniques 17: 310-315).

A method for generating a large population of multiple mutants using random in vivo recombination has also been described (Caren et al. (1994) Bio/Technology 12: 517-520). However, this method requires the recombination of two different libraries of plasmids, each library having a different selectable marker. Thus, the method is limited to a finite number of recombinations equal to the number of selectable markers existing and produces a concomitant linear increase in the number of marker genes linked to the selected sequence(s).

In vivo recombination between two homologous but truncated insect-toxin genes on a plasmid have been reported as also being capable of producing a hybrid gene (Calogero et al. (1992) FEMS Microbiology Lett. 76: 41-44; Galizzi et al. WO91/01087). The in vivo recombination of substantially mismatched DNA sequences in a host cell having defective mismatch repair enzymes, which results in hybrid molecule formation, has been reported (Radman et al. WO90/07576).

As discussed above, prior methods for producing random proteins from randomized genetic material have met with limited success. One method for producing and screening a wide variety of random proteins is a method which utilizes enzymes to cleave (chop) a long nucleotide chain into shorter pieces followed by procedures to separate the chopping agents from the genetic material and procedures to amplify (multiply the copies of) the remaining genetic material in a manner that allows the annealing of the polynucleotides back into chains (either purposefully or randomly put them back together). (Stemmer, Proc Natl Acad Sci USA (1994), 91:10747-10751; Stemmer, Nature 370 (1994) 389-391, U.S. Pat. Nos. 5,605,793, 5,811,238, 5,830,721, 5,928,905, 6,096,548, 6,117,679, 6,165,793, 6,153,410). Another method uses primers and limited polymerase extensions to generate the fragments prior to reassembly (U.S. Pat. Nos. 5,965,408, 6,159,687).

A drawback to this method is the likelihood of regenerating the parental template polynucleotides due to annealing of complementary single-strands from a particular parental template. Such regenerated parental molecules represent a background in the library of unchanged polynucleotides that can increase the difficulty of detecting recombinant molecules. This problem becomes increasingly severe as the percentage of sequence identity between the target parental templates decreases. With increasing sequence divergence, there is an increasing tendency for complementary parental template fragments to anneal, at some point resulting in the inability to detect any recombinant polynucleotides at all. Kikuchi, et al., (Gene 236:159-167 (1999)) attempted to generate recombinants between xylE and nahH by the methods of family shuffling (Patten et al., 1997; Crameri et al., 1998; Harayama, 1998; Kumamaru et al., 1998; Chang et al., 1999; Hansson et al., 1999), but failed to find any recombinants (<1%).

Accordingly, there is a need in the art for producing an improved method of forcing recombination among input parental templates such that reassembly produces randomized polynucleotides which can be screened for a particular use. The need to produce large libraries of widely varying mutant nucleic acid sequences is an important goal. Hence, it would be advantageous to develop such a method for the production of mutant proteins which allows for the development of large libraries of mutant nucleic acid sequences which are easily searched.

Thus, there exists a need to develop a method which allows for the production of large libraries of mutant DNA, RNA or proteins and the selection of particular mutants for a desired goal. The present invention satisfies this need and provides related advantages as well.

### SUMMARY OF THE INVENTION

The invention provides methods of forcing recombination between polynucleotides. The methods can include the steps of, generating a single strand of a first polynucleotide; generating a single strand of a second polynucleotide, wherein the second polynucleotide is partially complementary to the first polynucleotide; fragmenting the single strand of the first polynucleotide to generate single stranded first polynucleotide fragments; fragmenting the single strand of the second polynucleotide to generate single stranded second polynucleotide fragments; annealing the single stranded first polynucleotide fragments with the single stranded second polynucleotide fragments; and extending the annealed polynucleotide fragments.

### BRIEF DESCRIPTION OF THE DRAWINGS

FIG. 1 shows a diagram representing the fragmentation of a population of similar polynucleotides (A), and of a population of similar polynucleotides (B) having partial complementarity to A.

FIG. 2 shows a mixture of size-fractionated fragments from population A and size-fractionated fragments from population B. The fragments are allowed to anneal such that fragments of population A form overlapping partially double-stranded polynucleotides with fragments from population B. The annealed fragments are extended resulting in recombined output fragments.

FIG. 3 The output fragments from FIG. 2 can be denatured, annealed, and extended to form larger output fragments.

FIG. 4 shows a comparison of shuffling by the standard method in which both strands are present as templates (illustrated on the left side of the figure), and the current invention in which only a single strand of each parental template is introduced (illustrated on the right side of the figure). The standard method allows reassembly of fragments from a parental template. The present invention forces recombination in the first annealing and extension.

FIG. 5 shows an exemplary partially complementary nucleic acid population of two molecules. FIG. 5A shows the sequence of two nucleic acid molecules “X” and “Y” having completely complementary top/bottom strands 1+/2−(SEQ ID NOS:1 and 2) and 3+/4−(SEQ ID NOS:3 and 4), respectively. The positions of differing nucleotides between the nucleic acids X and Y are indicated (*). FIG. 5B shows possible combinations of single strands derived from nucleic acids X and Y after denaturing and annealing and indicates which of those combinations would comprise a partially complementary nucleic acid population of two.

FIG. 6 shows a graphic representation of the DNA sequences of the recombinants obtained by forcing recombination between TMV and TOMV movement proteins. The thin lines represent sequences most similar to TMV, and the thick lines represent sequences most similar to ToMV. The approximate scale in nucleotides is shown on the bottom. The 5′ ends of the gene coding sequences are on the left and the 3′ ends are on the right.

### DETAILED DESCRIPTION OF THE INVENTION

The present invention is directed to a method for generating a selected mutant polynucleotide sequence (or a population of selected polynucleotide sequences) typically in the form of amplified and/or cloned polynucleotides, whereby the selected polynucleotide sequences(s) possess at least one desired phenotypic characteristic (e.g., encodes a polypeptide, promotes transcription of linked polynucleotides, binds a protein, improves the function of a viral vector, and the like) which can be selected or screened for. Such desired polynucleotides can be used in a number of ways such as expression from a suitable plant, animal, fungal, yeast, or bacterial expression vector, integration to form a transgenic plant, animal or microorganism, expression of a ribozyme, and the like.

The invention described herein uses a single strand of each input parental template to force recombination in order for double-stranded products to result. The invention is directed to the use of repeated cycles of mutagenesis, recombination and selection, which allow for the directed molecular evolution of highly complex linear sequences, such as DNA, RNA or proteins through random recombination.

The methods of the invention advantageously allow the highly efficient production of recombinant nucleic acids, which can be used to screen for improved properties. The methods are based on forcing recombination between related polynucleotide templates during the process of assembling large polynucleotides from component polynucleotides.

DNA shuffling is a powerful method for evolving gene sequences in a directed manner to obtain recombinants between two or more DNA sequences. The products of DNA shuffling represent a pool of essentially random reassortments of gene sequences from the parental input DNAs. If the parental DNAs are various mutants of a given gene that have some improved characteristic over the wild-type, then the progeny DNAs of DNA shuffling will contain these beneficial mutations in novel combinations. These progeny molecules with novel combinations of beneficial mutations can then be analyzed for additive or synergistic effects resulting from new combinations of the individual mutations.

The method of DNA shuffling can be analogized to an evolutionary path in which only mutants with suitable properties are allowed to contribute their genetic material to production of the next generation. In this way, the path to ultimate genetic improvement is much more direct than it would be if random mutagenesis were used to generate the final product.

For example, if a sum of different point mutations were required to produce a mutant protein that has some desired set of enhanced properties, then one would have to generate a very large number of random mutants to get the one that has all of the desired changes and no deleterious mutations. If, instead, a more incremental change in protein performance can be discerned, several mutant genes can be selected from a mutagenized pool, each having a subset of the mutations that may together confer optimal performance to an encoded protein. These mutants can be recombined using DNA shuffling to obtain recombinants that perform better than their parents. Additional rounds of selection and shuffling can be used to eventually lead to the generation of a nucleic acid that contains an optimized set of mutations for a desired function of the nucleic acid or encoded polypeptide.

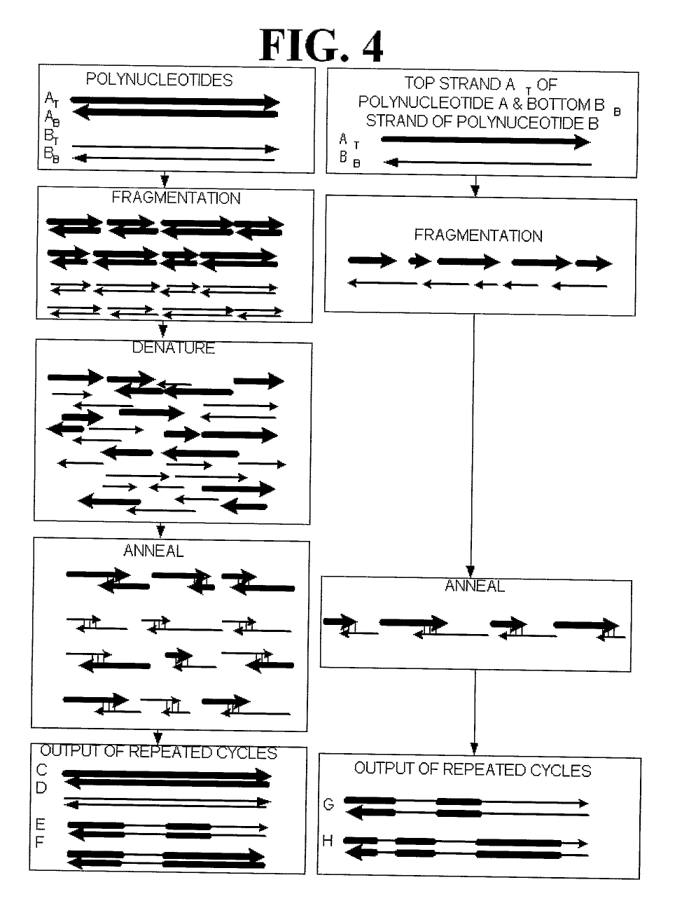

The present invention is advantageous in that a greater efficiency of recombinant nucleic acids can be achieved than by previously described methods. By including only one strand of each of the shuffling participants, the outcome of the shuffling technique can be controlled such that essentially all of the resulting PCR products are recombinant. In contrast, previously described gene shuffling methods allowed for reconstruction of any of the parental DNA molecules in the course of shuffling (see FIG. 4). Therefore, the methods of the present invention are advantageous in that the resulting plurality of recombinant polynucleotides produced by methods of the invention have a higher percentage of recombinants and therefore provides a lower background for identifying a desirable property of a mutant in the population. Furthermore, using previously described methods, the tendency toward parental reconstruction is expected to increase as a function of the level of diversity between the gene sequences to be shuffled. In contrast, the present invention essentially forces recombination of more distantly related nucleic acids. Therefore, the present invention is particularly useful for “molecular breeding,” where diverse, but related, sequences, for example, homologs from different species, are shuffled together. The present invention provides a much more efficient method for shuffling diverse sequences than previously described methods, as well as for shuffling highly similar sequences.

The invention provides a method of forcing recombination between polynucleotides. The method includes the steps of (a) generating a single strand of a first polynucleotide; (b) generating a single strand of a second polynucleotide, wherein the second polynucleotide is partially complementary to the first polynucleotide; (c) fragmenting the single strand of the first polynucleotide to generate single stranded first polynucleotide fragments;(d) fragmenting the single strand of the second polynucleotide to generate single stranded second polynucleotide fragments; (e) annealing the single stranded first polynucleotide fragments with the single stranded second polynucleotide fragments; and (f) extending the annealed polynucleotide fragments.

The method for producing polynucleotides can optionally further comprise the step of amplifying the double stranded polynucleotides of step (f). An optional step of isolating a desired size range of extended polynucleotides before amplification can be added. Additionally, the method can optionally further comprise the step of repeating steps (e) through (f) one or more times, wherein the double stranded polynucleotides resulting from step (f) are denatured prior to repeating steps (e) through (f).

The term “agent” is used herein to denote a chemical compound, a mixture of chemical compounds, an array of spatially localized compounds (e.g., a VLSIPS peptide array, polynucleotide array, and/or combinatorial small molecule array), biological macromolecule, a bacteriophage peptide display library, a bacteriophage antibody (e.g., scFv) display library, a polysome peptide display library, or an extract made form biological materials such as bacteria, plants, fungi, or animal (particular mammalian) cells or tissues. Agents are evaluated for potential activity as anti-neoplastics, anti-inflammatories or apoptosis modulators by inclusion in screening assays described hereinbelow. Agents are evaluated for potential activity as specific protein interaction inhibitors (i.e., an agent which selectively inhibits a binding interaction between two predetermined polypeptides but which does not substantially interfere with cell viability) by inclusion in screening assays described hereinbelow.

The term “amplification” means that the number of copies of a polynucleotide is increased.

As used herein, “annealing” refers to the formation of at least partially double stranded nucleic acid by hybridization of at least partially complementary nucleotide sequences. A partially double stranded nucleic acid can be due to the hybridization of a smaller nucleic acid strand to a longer nucleic acid strand, where the smaller nucleic acid is 100% identical to a portion of the larger nucleic acid. A partially double stranded nucleic acid can also be due to the hybridization of two nucleic acid strands that do not share 100% identity but have sufficient homology to hybridize under a particular set of hybridization conditions.

As used herein the term “clamps” refers to terminal extensions of unique sequences from the genes that are to be shuffled. The use of clamps in DNA shuffling ensures that only recombinant molecules can be amplified from the gene reassembly reaction. For example, if two genes are to be shuffled, one sequence clamp is incorporated at the 5′ extremity of one of the genes and another clamp, with a different sequence, is incorporated at the 3′ end of the other gene. The primers that will be used to amplify the product of the gene reassembly step of DNA shuffling anneal to each of the clamp regions. Since neither of the original starting genes had both primer binding sequences (clamps), they are not subject to PCR amplification. The only molecules that can be amplified are molecules that result from recombination between genes containing individual clamp sequences. Thus, the use of clamps is a way to ensure that only recombinant molecules are amplified after gene reassembly. A similar application of this approach was recently reported by Skarfstad, et al (Jounal of Bacteriology, Vol 182, No. 11, p 3008-3016, June 2000) where they used unique flanking sequences on either end of the two genes that they shuffled to prevent the recovery of non-recombinant sequences that had simply become reassembled in the shuffling reaction.

The term “chimeric polynucleotide” means that the polynucleotide comprises regions which are wild-type and regions which are mutated. It can also mean the polynucleotide comprises wild-type regions from one polynucleotide and wild-type regions from another related polynucleotide.

The term “cleaving” means digesting the polynucleotide with enzymes or breaking the polynucleotide by physical or chemical methods.

The term “cognate” as used herein refers to a gene sequence that is evolutionarily and functionally related between species. For example but not limitation, in the human genome the human CD4 gene is the cognate gene to the mouse 3d4 gene, since the sequences and structures of these two genes indicate that they are highly homologous and both genes encode a protein which functions in signaling T cell activation through MHC class II-restricted antigen recognition.

As used herein, the term “complementarity-determining region” and “CDR” refer to the art-recognized term as exemplified by the Kabat and Chothia CDR definitions also generally known as supervariable regions or hypervariable loops (Chothia and Leks, J. Mol. Biol. 196:901-917 (1987); Clothia et al., Nature 342:877-883 (1989); Kabat et al., Sequences of Proteins of Immunological Interest (National Institutes of Health, Bethesda, Md.) (1987); and Tramontano et al., J. Mol. Biol. 215:175-182 (1990)). Variable region domains typically comprise the amino-terminal approximately 105-115 amino acids of a naturally-occurring immunoglobulin chain (e.g., amino acids 1-110), although variable domains somewhat shorter or longer are also suitable for forming single-chain antibodies.

An immunoglobulin light or heavy chain variable region consists of a “framework” region interrupted by three hypervariable regions, also called CDR's. The extent of the framework region and CDR's have been precisely defined (see, oSequences of Proteins of Immunological Interest,” E. Kabat et al., 4th Ed., U.S. Department of Health and human services, Bethesda, Md. (1987)). The sequences of the framework regions of different light or heavy chains are relatively conserved within a species. As used herein, a “human framework region” is a framework region that is substantially identical (about 85% or more, usually 90-95% or more or even 99% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen.

As used herein, the phrase “comprises less than a recited percent identity of parental nucleic acids,” when used in reference to double stranded recombinant nucleic acid molecules, means that a plurality of double stranded polynucleotides contains less than 85% parental nucleic acid molecules. The plurality of double stranded recombinant polynucleotides can be, for example, less than about 84%, less than about 83%, less than about 82%, less than about 81%, less than about 80%, less than about 78%, less than about 75%, less than about 70%, less than about 60%, less than about 50%, less than about 40%, less than about 30%, less than about 20%, less than about 10%, less than about 5%, less than about 2%, or less than about 1%. Furthermore, a plurality of double stranded recombinant polynucleotides generated by a method of the invention can be less than about 0.5%, less than about 0.2%, less than about 0.1%, or can be a population containing essentially no parental nucleic acids.

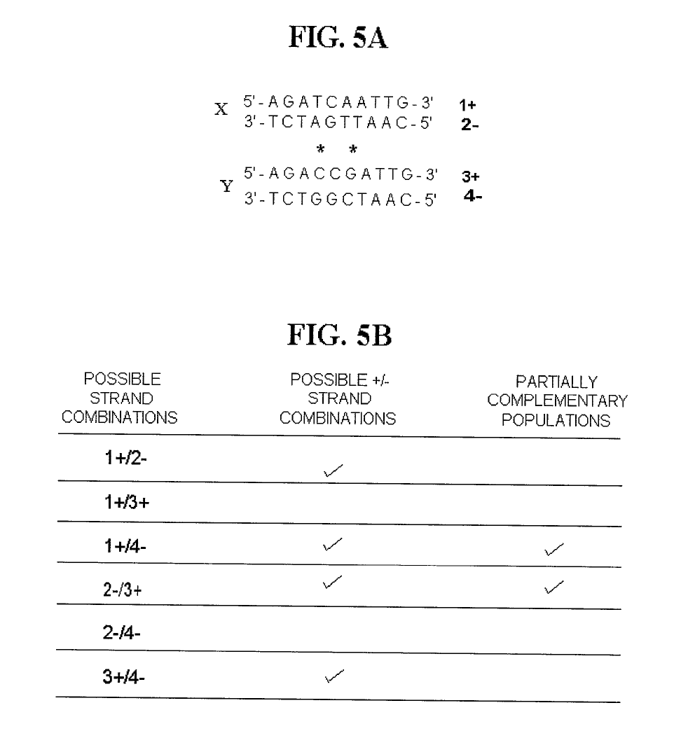

As used herein, the phrase “comprises sequences, or the complement thereto, having less than a recited % identity,” when used in reference to a partially complementary population of nucleic acids, refers to a starting population of single stranded nucleic acids in which members of the population, in either a top or bottom strand, have less than a specifically recited % identity. A population of two single stranded nucleic acids that are exactly complementary is a population containing nucleic acids comprising nucleotide sequences, or the complement thereto, having 100% identity. The exemplary partially complementary nucleic acid populations illustrated in FIG. 5 are representative of a population comprising sequences, or the complement thereto, having 80% identity. For example, a population can contain sequences, or the complement thereto, having less than 100% identity, that is, there is at least one nucleotide that differs between sequences of the same sense or the opposite sense. In an invention partially complementary population, the population contains sequences, or the complement thereto, that differ by at least one nucleotide. Accordingly, depending on the length of the nucleic acid molecules in the population, a population can contain sequences, or the complement thereto, having less than 100% identity, including less than 99.8% identity, for example, in the case of a 1000 nucleotide sequence differing by two nucleotides, or 99.98% identity in the case of a 10,000 nucleotide sequence differing by two nucleotides.

The invention thus provides a partially complementary nucleic acid population comprising sequences, or the complement thereto, having less than 100% identity, for example, less than about 99% identity, less than about 98% identity, less than about 97% identity, less than about 96% identity, less than about 95% identity, less than about 94% identity less than about 93% identity less than about 92% identity, less than about 91% identity, less than about 90% identity, less than about 85% identity, less than about 80% identity, or even less than about 75% identity.

The term “corresponds to” is used herein to mean that a polynucleotide sequence is homologous to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference “TATAC” and is complementary to a reference sequence “GTATA.”

As used herein, the term “defined sequence framework” refers to a set of defined sequences that are selected on a non-random basis, generally on the basis of experimental data or structural data; for example, a defined sequence framework can comprise a set of amino acid sequences that are predicted to form a β-sheet structure or can comprise a leucine zipper heptad repeat motif, a zinc-finger domain, among other variations. A “defined sequence kernal” is a set of sequences which encompass a limited scope of variability. Whereas (1) a completely random 10-mer sequence of the 20 conventional amino acids can be any of 20¹⁰ sequences, and (2) a pseudorandom 10-mer sequence of the 20 conventional amino acids can be any of 20¹⁰ sequences but will exhibit a bias for certain residues at certain positions and/or overall, (3) a defined sequence kernal is a subset of sequences if each residue position was allowed to be any of the allowable 20 conventional amino acids (and/or allowable unconventional amino/imino acids). A defined sequence kernal generally comprises variant and invariant residue positions and/or comprises variant residue positions which can comprise a residue selected from a defined subset of amino acid residues, and the like, either segmentally or over the entire length of the individual selected library member sequence. Defined sequence kernels can refer to either amino acid sequences or polynucleotide sequences. of illustration and not limitation, the sequences (NNK)₁₀ and (NNM)₁₀, wherein N represents A, T, G, or C; K represents G or T; and M represents A or C, are defined sequence kernels.

As used herein, “denaturing” or “denatured,” when used in reference to nucleic acids, refers to the conversion of a double stranded nucleic acid to a single stranded nucleic acid. Methods of denaturing double stranded nucleic acids are well known to those skilled in the art, and include, for example, increasing temperature, decreasing salt, or combinations thereof, depending on the % complementarity of the strands, that is, whether the strands are 100% complementary or have one or more non-complementary nucleotides.

The term “DNA shuffling” is used herein to indicate recombination between substantially homologous but non-identical sequences, in some embodiments DNA shuffling can involve crossover via non-homologous recombination, such as via cre/lox and/or flp/frt systems and the like.

As used herein “epitope” refers to that portion of an antigen or other macromolecule capable of forming a binding interaction that interacts with the variable region binding portion of an antibody. Typically, such binding interaction is manifested as an intermolecular contact with one or more amino acid residues of a CDR.

As used herein, “forced recombination” refers to using a set of reagents that cause recombination events which require one or more transfers of information between the top and bottom strands of a double stranded polynucleotide, because the polynucleotide is made by annealing and extending top and bottom strands that are partially complimentary and can be, for example, from different sources. When using forced recombination, 100% complimentarily of strands can only occur through information transfer from one strand to the other since none of the starting strands in the reaction have 100% complimentary. Information exchange is the reading of one strand to fill a gap in another strand, where the gap results from annealing of the strands. A non-recombination event would be the annealing of top and bottom strands to make the original parental polynucleotide without the need for information transfer. Non-recombination can occur where the starting reagents are the double stranded polynucleotide from each source.

The term “heterologous” means that one single-stranded nucleic acid sequence is unable to hybridize to another single-stranded nucleic acid sequence or its complement. Thus areas of heterology means that areas of polynucleotides or polynucleotides have areas or regions within their sequence which are unable to hybridize to another nucleic acid or polynucleotide. Such regions or areas are, for example areas of mutations.

The term “homologous” or “homeologous” means that one single-stranded nucleic acid nucleic acid sequence can hybridize to a complementary single-stranded nucleic acid sequence. The degree of hybridization can depend on a number of factors including the amount of identity between the sequences and the hybridization conditions such as temperature and salt concentrations, as discussed herein. Preferably the region of identity is greater than about 5 bp, more preferably the region of identity is greater than 10 bp.

The term “identical” or “identity” means that two nucleic acid sequences have the same sequence or a complementary sequence. Thus, “areas of identity” means that regions or areas of a polynucleotide or the overall polynucleotide are identical or complementary to areas of another polynucleotide or the polynucleotide.

As used herein “ligand” refers to a molecule, that is recognized by a particular receptor. A ligand can be, for example, a random peptide or variable segment sequence, as one of skill in the art will recognize, a molecule (or macromolecular complex) can be both a receptor and a ligand. In general, the binding partner having a smaller molecular weight is referred to as the ligand and the binding partner having a greater molecular weight is referred to as a receptor.

As used herein, “linker” or “spacer” refers to a molecule or group of molecules that connects two molecules, such as a DNA binding protein and a random peptide, and serves to place the two molecules in a preferred configuration, e.g., so that the random peptide can bind to a receptor with minimal steric hindrance from the DNA binding protein.

The term “mutations” means changes in the sequence of a wild-type nucleic acid sequence or changes in the sequence of a peptide. Such mutations can be point mutations such as transitions or transversions. The mutations can be deletions, insertions or duplications.

The term “naturally-occurring” as used herein as applied to the object refers to the fact that an object can be found in nature. For example, a polypeptide or polynucleotide sequence that is present in an organism (including viruses) that can be isolated from a source in nature and which has not been intentionally modified by man in the laboratory is naturally occurring. Generally, the term naturally occurring refers to an object as present in a non-pathological (un-diseased) individual, such as would be typical for the species.

As used herein, the term “nucleic acid” or “nucleic acid molecule” means a polynucleotide such as deoxyribonucleic acid (DNA) or ribonucleic acid (RNA) and encompasses both single-stranded and double-stranded nucleic acid as well as an oligonucleotide. Nucleic acids useful in the invention include genomic DNA, cDNA, mRNA and synthetic oligonucleotides corresponding thereto and can represent the sense strand, the anti-sense strand, or both. A nucleic acid generally incorporates the four naturally occurring nucleotides adenine, guanine, cytosine, and thymidine/uridine. An invention nucleic acid can also incorporate other naturally occurring or non-naturally occurring nucleotides, including derivatives thereof, so long as the nucleotide derivatives can be incorporated into a polynucleotide by a polymerase at an efficiency sufficient to generate a desired polynucleotide product.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

As used herein, a “parental nucleic acid” refers to a double stranded nucleic acid having a sequence that is 100% identical to an original single stranded nucleic acid in a starting population of partially complementary nucleic acids. Parental nucleic acids would include, for example, in the illustration of FIG. 5, nucleic acids X and Y if partially complementary nucleic acid combinations 1+/4− or 2−/3+ were used as a starting population in an invention method.

As used herein, “partially complementary” refers to a nucleic acid having a complementary sequence to another nucleic acid but that differs from the other nucleic acid by at least one nucleotide. As used herein, “partially complementary nucleic acid population” refers to a population of nucleic acids comprising nucleic acids having complementary sequences but no nucleic acids having an exact complementary sequence for any other member of the population. As used herein, an exact complement, when used in reference to a polynucleotide, refers to a polynucleotide having an exact complementary sequences, that is, 100% complementary, to another polynucleotide. As used herein, any member of a partially complementary nucleic acid population differs from another nucleic acid of the population, or the complement thereto, by one or more nucleotides. As such, a partially complementary nucleic acid specifically excludes a population containing sequences that are exactly complementary, that is, a complementary sequence that has 100% complementarity. Therefore, each member of such a partially complementary nucleic acid population differs from other members of the population by one or more nucleotides, including both strands. One strand is designated the top strand, and its complement is designated the bottom strand.

As used herein, “top” strand refers to a polynucleotide read in the 5′ to 3′ direction and the “bottom” its complement. It is understood that, while a sequence is referred to as bottom or top strand, such a designation is intended to distinguish complementary strands since, in solution, there is no orientation that fixes a strand as a top or bottom strand. In some instances, one strand can be a positive sense strand and the other strand is a minus sense strand, but in other instances, two complementary strands are both positive in that they encode a polypeptide. Some viruses are referred to as ambisense, that is, both strands contain protein-coding sequences.

For example, a population containing two nucleic acid members can be derived from two double stranded nucleic acids, with a potential of using any of the four strands to generate a single stranded partially complementary nucleic acid population. An example of potential combinations of strands of two nucleic acids that can be used to obtain a partially complementary nucleic acid population of the invention is shown in FIG. 5. The two nucleic acid sequences that are potential members of a partially complementary nucleic acid population are designated “X” (AGATCAATTG; SEQ ID NO:1) and “Y” (AGACCGATTG; SEQ ID NO:3)(FIG. 5A). The nucleic acid sequences differ at two positions (positions 3 and 5 indicated by “*”). The “top” strand of nucleic acids X and Y are designated “1+” and “3+,” respectively, and the “bottom” strand of nucleic acids X and Y are designated “2−” and “4−,” respectively.

FIG. 5B shows the possible combinations of the four nucleic acid strands. Of the six possible strand combinations, only the combination of 1+/2−, , 2−/3+, or 3+/4− comprise the required top and bottom 1+/4− strand of a partially complementary nucleic acid population. Of these top/bottom sequence combinations, only 1+/4− or 2−/3+ comprise an example of a partially complementary nucleic acid population of two different molecules because only these combinations have complementary sequences that differ by at least one nucleotide. The remaining combinations, 1+/2− and 2+/4− contain exactly complementary sequences and therefore do not comprise a partially complementary nucleic acid population of the invention.

In the above described example of a population of two different molecules, a partially complementary population of nucleic acid molecules excluded combinations of strands that differ by one or more nucleotides but which are the same sense, for example, 1+/3+ or 2−/4−. However, it is understood that such a combination of same stranded nucleic acids can be included in a larger population, so long as the population contains at least one bottom strand and at least one top strand. For example, if a third nucleic acid “Z,” with strands 5+and 6− is included, the combinations 1+/3+/6− or 2−/4−/5+ would comprise a partially complementary nucleic acid population. Similarly, any number of nucleic acids and their corresponding top and bottom strands can be combined to generate a partially complementary nucleic acid population of the invention so long as the population contains at least one top strand and at least one bottom strand and so long as the population contains no members that are the exact complement.

In an invention method, first polynucleotide can be a top or bottom strand. If a first polynucleotide is a top strand, then a second polynucleotide partially complementary to the first polynucleotide is a bottom strand. Similarly, if a first polynucleotide is a bottom strand, a second polynucletide partially complementary to the first strand is a top strand. If desired, an invention method can further include the use of polynucleotides in the opposite sense than in a first reaction of forcing recombination between polynucleotides. For example, if an invetnion method is performed using a first polynucleotide that is a top strand and a partially complementary secod strand, the steps of an invention mehtod can also be performed with a third polynucleotide that is the exact complement of the first polynucleotide and a fourth polynucloeotide that is the exact complement of the secod polynucleotide. The use of a second set of polynucleotides (third and fourth polynucleotides) exactly complementary to a first set of polynucleotides (first and secod polynucleotides) can be advantageous in that any bias of incorporated of mutations due to the use of one set of polynucleotides can be minimized.

For example, as illustrated in FIG. 5A, a first polynucleotide can be strand 1+ of nucleic acid “X”, and the partially complementary secod strand can be strand 4− of nucleic acid “Y”. If desired, a third polynucleotide that is the exact complement of strand 1+ can be used, that is, strand 2− can be used as a third polynucleotide. A fourth polynucleotide that is the exact compelment of strand 4− can be used, that is, strand 3+. Thus, two sets of reactions can be carried out, sequentially or in parallel, one containing first and secod polynucleotides that are partially complementary (1+ and 4−) and a secod reaction containing the exact complement of the first and second nucleotides (2− and 3+). The use of such parallel reactions can function to minimize any bias that occurs due to the use of one particular set of partially complementary polynucleotides for forcing recombination.

As used herein the term “physiological conditions” refers to temperature, pH, ionic strength, viscosity, and like biochemical parameters which are compatible with a viable organism, and/or which typically exist intracellularly in a viable cultured yeast cell, plant cell, or mammalian cell. For example, the intracellular conditions in a yeast cell grown under typical laboratory culture conditions are physiological conditions. Suitable in vitro reaction conditions for in vitro transcription cocktails are generally physiological conditions. In general, in vitro physiological conditions comprise 50-200 mM NaCl or KCl, pH 6.5-8.5, 20-45° C. and 0.001-10 mM divalent cation (e.g., Mg⁺⁺, Ca⁺⁺) preferably about 150 mM NaCl or KCl, pH 7.2-7.6, 5 mM divalent cation, and often include 0.01-1.0 percent nonspecific protein (e.g., BSA). A non-ionic detergent (Tween, NP-40, Triton X-100) can often be present, usually at about 0.001 to 2%, typically 0.05-0.2% (v/v). Particular aqueous conditions can be selected by the practitioner according to conventional methods. For general guidance, the following buffered aqueous conditions can be applicable: 10-250 mM NaCl, 5-50 mM Tris HCl, pH 5-8, with optional addition of divalent cation(s) and/or metal chelators and/or nonionic detergents and/or membrane fractions and/or anti-foam agents and/or scintillants.

As used herein, a “polymerase” refers to an enzyme that catalyzes the formation of polymers of nucleotides, that is, polynucleotides. A polymerase useful in the invention can be derived from any organism or source, including animal, plant, bacterial and viral polymerases. A polymerase can be a DNA polymerase, RNA polymerase, or a reverse transcriptase capable of transcribing RNA into DNA.

A “population of nucleic acids” refers to a group of 2 or more nucleic acids that differ by at least one or more nucleotides or, if the nucleic acids are of opposite sense, the complement of the nucleic acids differ by at least one or more nucleotides, that is, the population can contain partially complementary sequences so long as there is at least one non-complementary nucleotide between the sequences. The populations of the invention can therefore be about 3 or more, about 4 or more, about 5 or more, about 6 or more, about 7 or more, about 8 or more, about 9 or more, about 10 or more, about 12 or more, about 15 or more, about 20 or more, about 25 or more, about 30 or more, about 40 or more, about 50 or more, about 75 or more, about 100 or more, about 150 or more, about 200 or more, about 250 or more, about 300 or more, about 350 or more, about 400 or more, about 450 or more, about 500 or more, o r even about 1000 or more different nucleic acid molecules. A population can also contain about 2000 or more, about 5000 or more, about 1×10⁴ or more, about 1×10⁵ or more, about 1×10⁶ or more, about 5×10⁷ or more, or even about 1×10⁸ or more different nucleic acids. One skilled in the art can readily determine a desirable population to include in invention methods depending on the nature of the desired shuffling experiment outcome and the available screening methods, as disclosed herein.

As used herein, the term “pseudorandom” refers to a set of sequences that have limited variability, so that for example the degree of residue variability at another position, but any pseudorandom position is allowed some degree of residue variation, however circumscribed.

As used herein “random peptide library” refers to a set of polynucleotide sequences that encodes a set of random peptides, and to the set of random peptides encoded by those polynucleotide sequences, as well as any fusion proteins that contain those random peptides.

As used herein, “random peptide sequence” refers to an amino acid sequence composed of two or more amino acid monomers and constructed by a stochastic or random process. A random peptide can include framework or scaffolding motifs, which can comprise invariant sequences.

The term “reassembly” is used to indicate the process of putting fragments of polynucleotides together at regions of homology.

As used herein, “receptor” refers to a molecule that has an affinity for a given ligand. Receptors can be naturally occurring or synthetic molecules. Receptors can be employed in an unaltered state or as aggregates with other species. Receptors can be attached, covalently or non-covalently, to a binding member, either directly or via a specific binding substance. Examples of receptors include, but are not limited to, antibodies, including monoclonal antibodies and antisera reactive with specific antigenic determinants (such as on viruses, cells, or other materials), cell membrane receptors, complex carbohydrates and glycoproteins, enzymes, and hormone receptors.

As used herein, a “recombinant polynucleotide” refers to a polynucleotide that has undergone a recombination of at least two different nucleic acids from a starting population of parental single stranded nucleic acids.

The term “related polynucleotides” means that regions or areas of the polynucleotides are identical and regions or areas of the polynucleotides are heterologous.

The following terms are used to describe the sequence relationships between two or more polynucleotides: “reference sequence,” “comparison window,” “sequence identity,” “percentage of sequence identity,” and “substantial identity.”

A “reference sequence” is a defined sequence used as a basis for a sequence comparison; a reference sequence can be a subset of a larger sequence, for example, as a segment of a full-length cDNA or gene sequence given in a sequence listing, or can comprise a complete cDNA or gene sequence. Generally, a reference sequence is at least 20 nucleotides in length, frequently at least 25 nucleotides in length, and often at least 50 nucleotides in length. Since two polynucleotides can each (1) comprise a sequence (i.e., a portion of the complete polynucleotide sequence) that is similar between the two polynucleotides and (2) can further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window,” as used herein, refers to a conceptual segment of at least 20 contiguous nucleotide positions wherein a polynucleotide sequence can be compared to a reference sequence of at least 20 contiguous nucleotides and wherein the portion of the polynucleotide sequence in the comparison window can comprise additions or deletions (i.e., gaps) of 20 percent or less as compared to the reference sequence (which does not comprise additions or deletions) for optimal alignment of the two sequences. Optimal alignment of sequences for aligning a comparison window can be conducted by the local homology algorithm of Smith and Waterman (Adv. Appl. Math. 2:482 (1981)), by the homology alignment algorithm of Needleman and Wunsch (J. Mol. Biol. 48:443 (1970)), by the search of similarity method of Pearson and Lipman (Proc. Natl. Acad. Sci. U.S.A. 85:2444 (1988)), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. This “substantial identity” as used herein denotes a characteristic of a polynucleotide sequence, wherein the polynucleotide comprises a sequence having at least 80 percent sequence identity, preferably at least 85 percent identity, often 90 to 95 percent sequence identity, and most commonly at least 99 percent sequence identity as compared to a reference sequence of a comparison window of at least 25-50 nucleotides, wherein the percentage of sequence identity is calculated by comparing the reference sequence to the polynucleotide sequence, which can include deletions or additions which total 20 percent or less of the reference sequence over the window of comparison.

As used herein, the term “single-chain antibody” refers to a polypeptide comprising a V_{H} domain and a V_{L} domain in polypeptide linkage, generally linked via a spacer peptide, for example, (Gly-Gly-Gly-Gly-Ser)ₓ, and which can comprise additional amino acid sequences at the amino- and/or carboxy- termini. For example, a single-chain antibody can comprise a tether segment for linking to the encoding polynucleotide. As an example, a scfv is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts eds., (1989) Academic Press: San Diego, Calif., pp. 361-368, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope).

“Specific hybridization” is defined herein as the formation of hybrids between a first polynucleotide and a second polynucleotide (e.g., a polynucleotide having a distinct but substantially identical sequence to the first polynucleotide), wherein substantially unrelated polynucleotide sequences do not form hybrids in the mixture.

The term “specific polynucleotide” means a polynucleotide having certain end points and having a certain nucleic acid sequence. Two polynucleotides wherein one polynucleotide has the identical sequence as a portion of the second polynucleotide but different ends comprises two different specific polynucleotides.

As used herein, “substantially pure” means an object species is the predominant species present (i.e., on a molar basis it is more abundant than any other individual macromolecular species in the composition), and preferably substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. Solvent species, small molecules (<500 Daltons), and elemental ion species are not considered macromolecular species.

As used herein, the term “variable segment” refers to a portion of a nascent peptide which comprises a random, pseudorandom, or defined kernal sequence A variable segment can comprise both variant and invariant residue positions, and the degree of residue variation at a variant residue position can be limited: both options are selected at the discretion of the practitioner. Typically, variable segments are about 5 to 20 amino acid residues in length (e.g., 8 to 10), although variable segments can be longer and can comprise antibody portions or receptor proteins, such as an antibody fragment, a nucleic acid binding protein, a receptor protein, and the like.

The term “wild-type” means that the polynucleotide does not comprise any mutations. A “wild type” protein means that the protein will be active at a level of activity found in nature and will comprise the amino acid sequence found in nature.

In the polypeptide notation used herein, the left-hand direction is the amino terminal direction and the right-hand direction is the carboxy-terminal direction, in accordance with standard usage and convention. Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the coding RNA transcript are referred to as “downstream sequences”.

### Methodology

In methods of the invention, single stranded nucleic acid fragments are generated by fragmenting single stranded nucleic acids, for example, by enzymatic digestion, shearing, and the like. Methods of fragmenting single stranded nucleic acids are well known to those skilled in the art (see, for example, Sambrook et al., Molecular Cloning: A Laboratory Manual, 2nd ed., Cold Spring Harbor Press, Plainview, N.Y. (1989); Ausubel et al., Current Protocols in Molecular Biology (Supplement 47), John Wiley & Sons, New York (1999)).

Any of a variety of methods can be used to generate single stranded nucleic acid template molecules, for example, using asymmetric PCR, single stranded phage, amplifying nucleic acids with an affinity tag, generation of RNA, or blocking a nucleic acid to prevent enzymatic digestion. For asymmetric PCR, either a forward or a reverse primer is used for each nucleic acid to generate the single-stranded nucleic acid molecules. Preferably, asymmetric PCR (Peter C. McCabe. 1990. Production of single stranded DNA by asymmetric PCR in PCR Protocols: A guide to methods and applications. eds. M. A. Innis, D. H. Gelfand, J. J. Sninsky and T. J. White. Academic Press Inc. San Diego, Calif. USA.) is conducted so that there are minimal or no flanking sequences present which have significant homologies to one another.

Another method of generating single stranded nucleic acid is to use single stranded phage. For example, the nucleic acid of interest can be cloned into a phagemid so that, upon helper phage infection of E. coli that harbors the nucleic acid of interest, single-stranded phagemid DNA is produced, packaged, and secreted into the medium. The strandedness of the recovered DNA strand can be determined by the orientation of the phage origin of replication with respect to the gene of interest. Methods of generating single stranded nucleic acids using phage are well known to those skilled in the art (see Sambrook et al., supra, 1989; Ausubel et al., supra, 1999).

Still another method to generate single stranded nucleic acid is to synthesize nucleic acid with an affinity tag for one strand. For example, a PCR primer can be covalently linked with an affinity tag such as biotin or any appropriate affinity tag followed by PCR amplification with an un-modified primer as the other amplimer. The resulting PCR product would be biotinylated at one terminus on only one strand so that, after immobilizing the PCR product on an affinity support such as streptavidin, the un-biotinylated strand can be removed from the immobilized strand by melting and washing. Any suitable affinity tag can be incorporated into a primer so long as the tag does not interfere with any required synthesis reaction, for example, does not inhibit a polymerase such that a sufficient quantity of product cannot be produced.

Another method to produce single stranded nucleic acid is to make RNA. For example, RNA transcripts of a gene of interest can be generated such that only one strand is represented in each transcript.

Yet another method of generating single stranded nucleic acid is to end-fill a PCR product at one end with phosphorothioate dNTPs to block the action of an exonuclease. Exonuclease treatment eliminates the un-modified strand. An exemplary exonuclease is Exonuclease III, which has 3′-5′ activity.

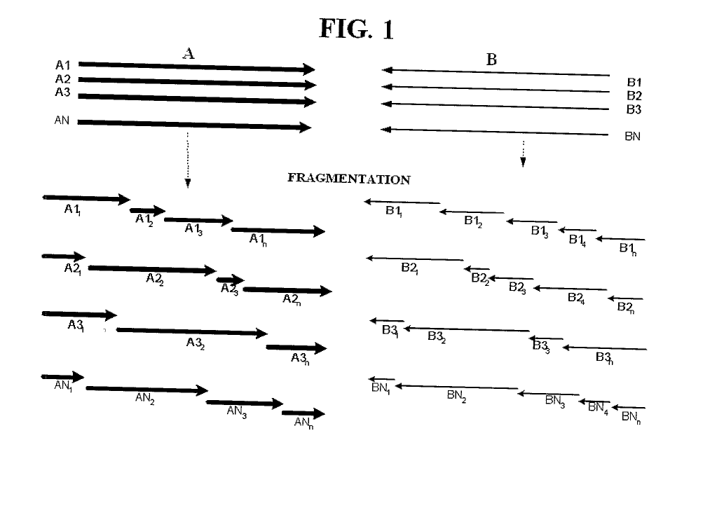

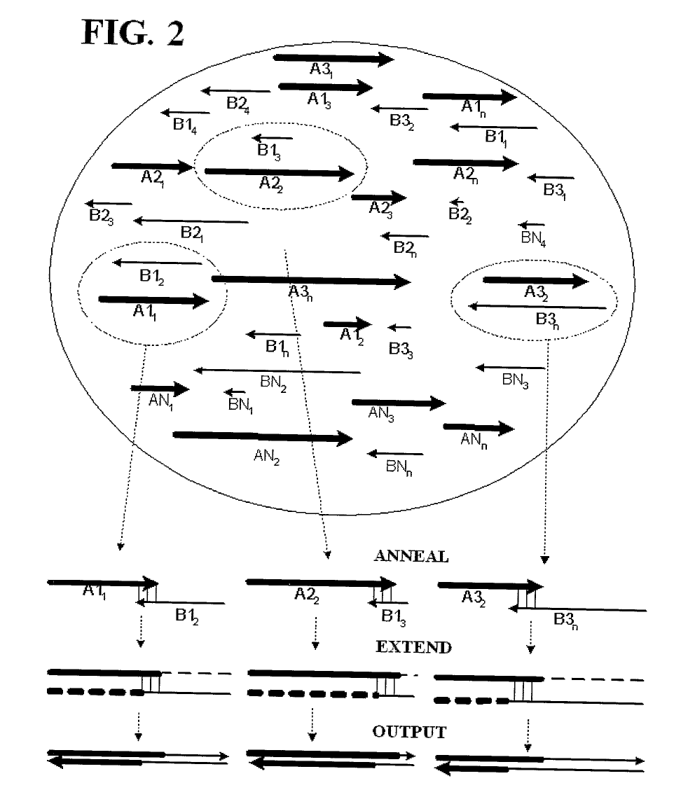

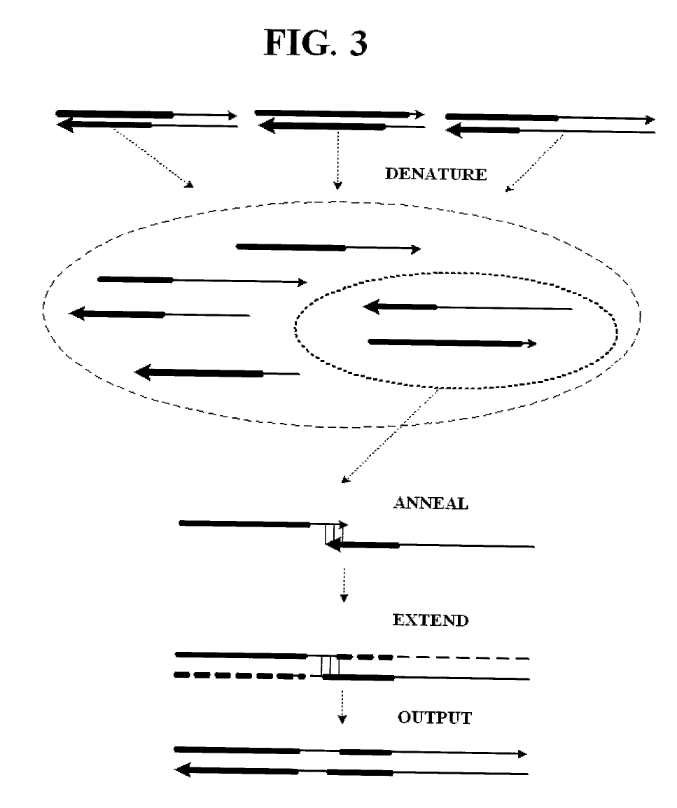

In another embodiment, the invention provides a process for producing mutant polynucleotides. The method includes the steps of generating single-stranded DNA (ssDNA) template for one or more polynucleotides by asymmetric PCR, generating the complementary ssDNA template of the polynucleotides differing by one or more nucleotides; fragmenting the ssDNA templates by a method such as nuclease treatment or shearing followed by isolation of fragments of appropriate size (FIG. 1); mixing the two pools of partially complementary ssDNA fragments; incubating the pools under conditions that result in the annealing of the single-stranded fragments at areas of identity to form pairs of annealed fragments; incubating under conditions which result in extension of the single-stranded polynucleotides at regions of identity between the single-stranded polynucleotides and thus forming a mutagenized double-stranded polynucleotide chain (FIG. 2). The method can optionally repeat the steps of annealing and extension after denaturation of the extended polynucleotides (FIG. 3). The method can further include the steps of expressing at least one mutant polypeptide from the polynucleotide chain, or chains; and testing the at least one mutant polypeptide for a useful activity. The method can further include the steps of screening or selection of polynucleotides conferring a desired property and subjecting these polynucleotides to the above method to generate recombinants with even more improved properties.

FIG. 1 shows a representation of the fragmentation of a library of one or more related single stranded polynucleotides of the same sense (Al through AN) to form top strand fragments. Each individual polynucleotide breaks into fragments (e.g., A1₁ through A1ₙ) fairly randomly giving rise to a population of overlapping fragments of related sequence. Similarly, a library of related single stranded polynucleotides partially complementary to the A polynucleotide(s) (B1 through BN) are fragmented to generate bottom strand fragments. At this point the fragments can be fractionated by size (e.g., by agarose gel electrophoresis). The size of the fragments chosen will affect the number of crossover events in the final recombinant molecules; larger fragments give fewer crossovers, while smaller fragments give more crossovers. Processing the A fragments separately from the B fragments affords the opportunity to isolate different size ranges of fragments for A and B if desired. In an alternative to the method above, the fragments of A and B could be mixed before size fractionation, or the input single stranded polynucleotides A and B could be mixed (with or without allowing annealing to occur) before fragmentation.

FIG. 2 illustrates the idea of mixing the fragments generated in FIG. 1 in a reaction that will allow annealing of fragments at regions of homology and extension of the partially double stranded polynucleotides by a polymerase to form recombined output fragments. Recombination is forced because no bottom strands from A polynucleotides were introduced, so in order to form a duplex an A (top strand) fragment must anneal to an appropriate B (bottom strand) fragment.

FIG. 3 illustrates the result of an additional round of annealing and extension. The fragments generated in FIG. 2 are denatured to single strands and then allowed to anneal. Some fragments will find new partners. Pairs with appropriate overlaps can be extended by the polymerase to form larger output fragments. Repeated rounds of denaturation, annealing and extension will result in full-length molecules. These molecules can be cloned (in all or in part) into a vector directly, or after addition of appropriate primers and amplification in a PCR reaction. Such clones can be tested by screening or selection for the desired properties. Clones with improved properties can be chosen as sources to generate input single stranded polynucleotides (as in FIG. 1) for an additional round of forced recombination.

FIG. 4 shows a contrast between previously described shuffling methods (shown on the left side of the Figure) and the present invention (shown on the right side of the Figure). In the previously described methods, both strands of an input template are used to generate fragments that are introduced into the reaction. After renaturation and reannealing, the top and bottom strands of a particular parent polynucleotide can come back together. With increasing diversity of input sequences to be shuffled, such as in molecular breeding, there will be an increasing fraction of output fragments that are non-recombined or have sequence identical to a parental input fragment (output polynucleotides C and D). In contrast, with the present invention, since recombination is forced in the first round of annealing and extension, although the absolute number of output clones can be smaller than with previously described methods, the fraction of output clones with sequence different than any parental input polynucleotide will be higher. In the limiting case of shuffling divergent sequences, the previously described methods may be unable to sort through enough output clones to detect any non-parental type output polynucleotides, while the present invention will allow recombinants to be obtained if such recombination can occur at all (e.g., output fragments G or H).

An additional embodiment provides a method of forcing recombination between polynucleotides, comprising: (a) fragmenting a single stranded first polynucleotide and a single stranded second polynucleotide to generate single stranded first and second polynucleotide fragments, wherein the second polynucleotide is partially complementary to the first polynucleotide, and optionally isolating a size range of single stranded fragments; (b) annealing the single stranded first polynucleotide fragments with the single stranded second polynucleotide fragments; and (c) extending the annealed fragments. The method can optionally further comprise adding at least one additional single stranded polynucleotide partially complementary to the first or second polynucleotide at step (a). The method for producing polynucleotides can optionally further comprise the step of amplifying the double stranded polynucleotides of step (c). Additionally, the method can optionally further comprise the step of repeating steps (b) through (c) one or more times, wherein the double stranded polynucleotides resulting from step (c) are denatured prior to repeating steps (b) through (c).

In another embodiment, the initial single strand input polynucleotides are relatively divergent (e.g., related genes from different organisms), and fragments of an additional one or more input single strand polynucleotide(s) of closely related sequence to one of the initial input polynucleotides is added after the first (or first few) rounds of annealing, extension and denaturation. In this way, recombination can be forced between the most distantly related sequences before more closely related sequences are introduced.

An additional embodiment provides a method of forcing recombination between polynucleotides, comprising: (a) generating a single strand of a first polynucleotide; (b) generating a single strand of a second polynucleotide, wherein the second polynucleotide is partially complementary to the first polynucleotide; (c) annealing the single strands of the first and second polynucleotides; (d) fragmenting the annealed polynucleotides to generate partially double stranded polynucleotide fragments, and optionally isolating a size range of single stranded fragments; (e) denaturing the partially double stranded polynucleotide fragments; (f) annealing the denatured polynucleotide fragments; and (g) extending the annealed polynucleotide fragments. The method can optionally further comprise adding at least one additional single stranded polynucleotide partially complementary to the first or second polynucleotide at step (c). The method for producing polynucleotides can optionally further comprise the step of amplifying the double stranded polynucleotides of step (g). Additionally, the method can optionally further comprise the step of repeating steps (e) through (g) one or more times.

It is understood that the steps of invention methods can be performed in any order so long as a desirable polynucleotide product is generated. Thus, the method steps can be performed in any order, and the steps can be performed sequentially, in parallel, or simultaneously, that is, in the same reaction vessel, as desired. One skilled in the art can readily determine an appropriate order of steps of invention methods as well as appropriate steps that can be performed sequentially, in parallel, or simultaneously that are sufficient to generate a desirable polynucleotide product.

Furthermore, invention method steps can be performed in any desirable order. For example, the methods can be performed by generating single stranded polynucleotides, which are fragmented, annealed and extended. Alternatively, the methods can be performed by annealing single stranded polynucleotides and fragmenting the annealed polynucleotides to generate partially double stranded polynucleotides. The invention methods can also be performed with various combinations of additional polynucleotides, which can also be performed sequentially, in parallel or simultaneously. For example, as disclosed herein, an initial shuffling reaction can be performed with two partially complementary polynucleotides, and subsequently shuffled with a third polynucleotide., Alternatively, multiple polynucleotides can simultaneously be shuffled in the same reaction.

The present invention will be of particular use importance in instances for which efficiency is important. If the process for identifying improved mutants involves a screen, each regenerated parental molecule screened represents wasted resources, so the present invention, in generating only recombinant full-length polynucleotides, will be more efficient.

A method for enhancing the efficiency of recombination during DNA family shuffling has been described (Kikuchi, et al., Gene 243:133-137 (2000)). Though it was found to be more efficient than previously described methods of family shuffling (Stemmer, Proc. Natl. Acad. Sci. USA 91:10747-10751 (1994)), only 14% of the clones that were recovered were recombinants. The methods disclosed herein are advantageous in that the methods provide a population of polynucleotides that can be essentially 100% recombinants.

The present invention relates to an enhanced method of DNA “shuffling,” which can be referred to as “heterosexual PCR.” Heterosexual PCR means that PCR amplification is used to generate a recombinant population of nucleic acids where each of the single stranded nucleic acids of the starting population each differ from other single stranded nucleic acids, or the complement thereto, by at least one nucleotide. optionally, the method comprises the additional step of testing the library members of the shuffled pool to identify individual shuffled library members having the ability to bind or otherwise interact (e.g., such as catalytic antibodies) with a predetermined macromolecule, such as, for example, a proteinaceous receptor, peptide oligosaccharide, virion, or other predetermined compound or structure.

The displayed polypeptides, antibodies, peptidomimetic antibodies, and variable region sequences that are identified from such libraries can be used for therapeutic, diagnostic, research and related purposes (e.g., catalysts, solutes for increasing osmolarity of an aqueous solution, and the like), and/or can be subjected to one or more additional cycles of shuffling and/or affinity selection. The method can be modified such that the step of testing for a phenotypic characteristic can be other than of binding affinity for a predetermined molecule (e.g., for catalytic activity, stability oxidation resistance, drug resistance, or detectable phenotype conferred upon a host cell).

In one embodiment, the first plurality of chosen library members is polynucleotides produced and homologously recombined by PCR in vitro, the resultant polynucleotides are transferred into a host cell or organism via a transferring means and homologously recombined to form shuffled library members in vivo.

In one embodiment, the first plurality of chosen library members is cloned or amplified on episomally replicable vectors, a multiplicity of said vectors is transferred into a cell and homologously recombined to form shuffled library members in vivo.

In one embodiment, the first plurality of chosen library members is not produced as shorter or smaller polynucleotides, but is cloned or amplified on a episomally replicable vector as a direct repeat, with each repeat comprising a distinct species of chosen library member sequence, said vector is transferred into a cell and homologously recombined by intra-vector recombination to form shuffled library members in vivo.

In an embodiment, combinations of in vitro and in vivo shuffling are provided to enhance combinatorial diversity.

The present invention provides a method for generating libraries of displayed antibodies suitable for affinity interactions screening. The method comprises (1) obtaining first a plurality of selected library members comprising a displayed antibody and an associated polynucleotide encoding the displayed antibody, and obtaining the associated polynucleotide encoding for the displayed antibody and obtaining the associated polynucleotides or copies thereof, wherein the associated polynucleotides comprise a region of substantially identical variable region framework sequence, and (2) pooling and producing shorter or smaller polynucleotides with the associated polynucleotides or copies to form polynucleotides under conditions suitable for PCR amplification by slowing or halting the PCR amplification and thereby homologously recombining the shorter or smaller polynucleotides to form a shuffled pool of recombined polynucleotides of the shuffled pool. CDR combinations are generated by the shuffled pool that are not present in the first plurality of selected library members, the shuffled pool composing a library of displayed antibodies comprising CDR permutations and suitable for affinity interaction screening. Optionally, the shuffled pool is subjected to affinity screening to choose shuffled library members which bind to a predetermined epitope (antigen) and thereby selecting a plurality of selected shuffled library members. Further, the plurality of selectedly shuffled library members can be shuffled and screened iteratively, from 1 to about 1000 cycles or as desired until library members having a desired binding affinity are obtained.

Another aspect of the present invention is directed to a method for obtaining chimeric polynucleotides by treating a sample comprising different single-stranded template polynucleotides wherein the different template polynucleotides contain areas of identity and areas of heterology under heterosexual PCR conditions which provide random single-stranded polynucleotides of a desired size from the template polynucleotide; incubating the resulting single-stranded polynucleotides with a polymerase under conditions which provide for the annealing of the single-stranded polynucleotides at the areas of identity and the formation of a chimeric double-stranded polynucleotide sequence comprising template polynucleotide sequences; and repeating the above steps as desired.

The invention also provides the use of polynucleotide shuffling to shuffle polynucleotides encoding polypeptides and/or polynucleotides comprising transcriptional regulatory sequences.

The invention also provides the use of polynucleotide shuffling to shuffle a population of viral genes (e.g., capsid proteins, spike glycoproteins, polymerases, proteases, and the like) or viral genomes (e.g., paramyxoviridae, orthomyxoviridae, herpesviruses, retroviruses, reoviruses, rhinoviruses, tobacco mosaic virus and the like). In an embodiment, the invention provides a method for shuffling sequences encoding all or portions of immunogenic viral proteins to generate novel combinations of epitopes as well as novel epitopes created by recombination; such shuffled viral proteins can comprise epitopes or combinations of epitopes as well as novel epitopes created by recombination; such shuffled viral proteins can comprise epitopes or combinations of epitopes which are likely to arise in the natural environment as a consequence of viral evolution; (e.g., such as recombination of influenza virus strains).

The invention also provides a method suitable for shuffling polynucleotide sequences for generating gene therapy vectors and replication-defective gene therapy constructs, such as can be used for human gene therapy, including but not limited to vaccination vectors for DNA-based vaccination, as well as anti-neoplastic gene therapy and other general therapy formats.

The methods of the invention can be used to test for optimized characteristics of a nucleic acid or a polypeptide encoded by a nucleic acid, such as DNA viruses (see U.S. Pat. No. 6,096,548) and also RNA viruses. For example, the methods of the invention can be used to optimize characteristics of a virus or viral vector (see Examples 2, 3 and 4).

The present invention relates to a method for nucleic acid molecule reassembly after producing random oligonucleotides via interrupted PCR, and optionally subjecting at least one of the random oligonucleotides to further PCR as templates to produce additional oligonucleotides, and the application of such reassembly to mutagenesis of DNA sequences. Also described is a method for the production of polynucleotides encoding mutant proteins having enhanced biological activity. In particular, the present invention also relates to a method of utilizing repeated cycles of mutagenesis, nucleic acid shuffling according to the present invention heterosexual PCR oligonucleotide method and screening of selection which allow for the creation of mutant proteins having enhanced biological activity.

The present invention is directed to a method for generating a very large library of DNA, RNA or protein mutants. This method has particular advantages in the generation of related polynucleotides from which the desired active polynucleotide portion(s) can be chosen. In particular, the present invention also relates to a method of repeated cycles of mutagenesis, homologous recombination and screening or selection which allow for the creation of mutant proteins having enhanced biological activity.

Nucleic acid shuffling is a method for in vitro or in vivo homologous recombination of pools of shorter or smaller polynucleotides to produce a polynucleotide or polynucleotides. Mixtures of related nucleic acid sequences or polynucleotides are subjected to heterosexual PCR to provide random polynucleotides, and reassembled to yield a library or mixed population of recombinant mutant nucleic acid molecules or polynucleotides.

In contrast to cassette mutagenesis, only shuffling and error-prone PCR allow one to mutate a pool of sequences blindly (without sequence information other than primers)

This method differs from error-prone PCR, in that it is an inverse chain reaction. In error-prone PCR, the number of polymerase start sites and the number of molecules grows exponentially. However, the sequence of the polymerase start sites and the sequence of the molecules remains essentially the same. In contrast, in nucleic acid reassembly or shuffling of random polynucleotides, the number of start sites and the number (but not size) of the random polynucleotides decreases over time. For polynucleotides derived from whole plasmids the theoretical endpoint is a single, large concatemeric molecule.

Rare shufflants will contain a large number of the best mutations, and these rare shufflants can be selected based on their superior characteristics.

Mutations from a pool of 100 different selected sequences can be permutated in up to 100⁶ different ways. This large number of permutations cannot be represented in a single library of DNA sequences. Accordingly, it is contemplated that multiple cycles of DNA shuffling and selection can be required depending on the length of the sequence and the sequence diversity desired.

Error-prone PCR, in contrast, keeps all the selected mutations in the same relative sequence, generating a much smaller mutant cloud.

The polynucleotide used in the methods of this invention can be DNA or RNA. The polynucleotides can be of various lengths depending on the size of the gene or shorter or smaller polynucleotide to be recombined or reassembled. Preferably, the polynucleotide is from 50 bp to 50 kb. It is contemplated that entire vectors containing the nucleic acid encoding the protein of interest can be used in the methods of this invention, and in fact have been successfully used.

The polynucleotide can be obtained by amplification using the PCR reaction (U.S. Pat. Nos. 4,683,202 and 4,683,195) or other amplification or cloning methods. However, the removal of free primers from the PCR products before subjecting them to pooling of the PCR products and heterosexual PCR can provide more efficient results. Failure to adequately remove the primers from the original pool before heterosexual PCR can lead to a low frequency of crossover clones.

The original polynucleotides should not be double-stranded. A single-stranded nucleic acid molecule is recommended to ensure that any full-length molecules resulting from the reassembly reaction must have undergone a recombination event.

Preferably, no regions of homology between the polynucleotides exist in regions that flank the sequences that are to be shuffled. Inclusion of such flanking regions of homology can lead to a dramatic reduction of efficiency of a disclosed method.

The single-stranded polynucleotide(s) are fragmented to provide a mixture of from about 5 bp to 5 kb or more. Preferably the size of the random polynucleotides is from about 10 bp to 1000 bp, more preferably the size of the polynucleotides is from about 20 bp to 500 bp.

The nucleic acid fragment can be digested by a number of different methods. The nucleic acid fragment can be digested with a nuclease, such as DNAseI or RNAse. The nucleic acid can be randomly sheared by the method of sonication or by passage through a tube having a small orifice.

It is also contemplated that the nucleic acid can also be partially digested with one or more restriction enzymes, such that certain points of crossover are retained statistically.

Single-stranded or double-stranded polynucleotides, either synthetic or natural, can be added to the random single-stranded shorter or smaller polynucleotides in order to increase the heterogeneity of the mixture of polynucleotides. It is also contemplated that populations of single-stranded randomly broken polynucleotides can be mixed or combined with the polynucleotides from the heterosexual PCR process and optionally subjected to one or more additional PCR cycles.

Where insertion of mutations into the template polynucleotide is desired, single-stranded or double-stranded polynucleotides having a region of identity to the template polynucleotide and a region of heterology to the template polynucleotide can be added in a 20 fold excess by weight as compared to the total nucleic acid, more preferably the single-stranded polynucleotides can be added in a 10 fold excess by weight as compared to the total nucleic acid.

Where a mixture of different but related template polynucleotides is desired, populations of polynucleotides from each of the templates can be combined at various ratios, for example, a ratio of less than about 1:100, more preferably at a ratio of less than about 1:40. For example, a backcross of the wild-type polynucleotide with a population of mutated polynucleotides can be used to eliminate neutral mutations (e.g., mutations yielding an insubstantial alteration in the phenotypic property being selected for). In such an example, the ratio of randomly provided wild-type polynucleotides, which can be added to the randomly provided heterosexual PCR cycle mutant polynucleotides, is approximately 1:1 to about 100:1, and more preferably from 1:1 to 40:1.

The mixed population of random polynucleotides are denatured to reduce secondary structure in the single-stranded polynucleotides and then allowed to anneal. Only those single-stranded polynucleotides having regions of homology with other single-stranded polynucleotides will anneal.

The random polynucleotides can be denatured by heating. One skilled in the art can determine the conditions necessary to completely denature the nucleic acid. Preferably the temperature is from 80° C. to 100° C., more preferably the temperature is from 90° C. to 96° C. Other methods which can be used to denature the polynucleotides include pressure (Coelho-Sampaio (1993) Biochem. 32:10929-10935) and pH.

The polynucleotides can be annealed by cooling. Preferably the temperature is from 20° C. to 75° C., more preferably the temperature is from 40° C. to 65° C. If a high frequency of crossovers is needed based on an average of only 4 consecutive bases of homology, recombination can be forced by using a low annealing temperature, although the process becomes more difficult. The degree of renaturation which occurs will depend on the degree of homology between the population of single-stranded polynucleotides.

Renaturation can be accelerated by the addition of polyethylene glycol (“PEG”) or salt. The salt concentration is preferably from 0 mM to 200 mM, more preferably the salt concentration is from 10 mM to 100 mM. The salt can be KCl or NaCl. The concentration of PEG is preferably from 0% to 20%, more preferably from 5% to 10%.

The annealed polynucleotides are incubated in the presence of a nucleic acid polymerase and nucleoside triphosphates, for example, dNTP's (i.e. DATP, dCTP, dGTP and dTTP). The nucleic acid polymerase can be the Klenow fragment, the Taq polymerase or any other DNA polymerase known in the art. If RNA polynucleotides are synthesized, RNA polymerase can be used in the presence of NTP's.

The approach to be used for the assembly depends on the minimum degree of homology that should still yield crossovers. If the areas of identity are large, Taq polymerase can be used with an annealing temperature of between 45-65° C. If the areas of identity are small, Klenow polymerase can be used with an annealing temperature of between 20-30° C. One skilled in the art can vary the temperature of annealing to increase the number of crossovers achieved.

If desired, the reaction conditions for a shuffling reaction can be varied to obtain an optimized outcome. For example, the annealing temperature, time of extension, number of cycles, buffer composition, and the like, can be varied to obtain optimized conditions for production of recombinant polynecleotides using an inventionn method. When using a thermostable polymerase, the reaction is generally carried out between about 20° C. up to about 95° C., and the extension reaction is carried out at a temperature sufficient to allow synthesized double stranded polynucleotides to remain annelaed, in general using temperatures of about 35° C. to about 80° C., in particular about 45-75° C., about 50-75° C., about 50-60° C., about 60-70° C., or about 70-75° C. (see exemplary conditions described in the Examples.)

In addition to using thermostable polymerases for amplification reactions, a heat-labile polymerase such as Klenow fragment can be used to synthesize a polynecleotide (see, for example, U.S. Pat. No. 5,830,721, issued Nov. 3, 1998). For example, the reaction can be carried out with a heat-labile polymerase such as Klenow fragment at a reduced temperature such as about 15° C. to about 40° C., generally about 25° C. to about 37° C., for the polymerization reaction. Following the denauration step, the sample can be rapidly cooled, for example, using dry ice/ethanol. The heat-labile polymerase is added at every cycle since the temperature for denaturation of double stranded polynucleotides would also denature the heat-labile polymerase. The use of low temperature annealing is particularly useful to force efficient crossovers based on short regions of homology (U.S. Pat. No.: 5,830,721). If desired, a thermostable polymerase can be used in sequential cycles to cycles performed with a heat-labile polymerase, that is, a certain number of cycles can be performed in the presence of a heat-labile polymerase, for example, to force recombination, followed by a series of cycles with a thermostable polymerase to carry out additional cycles of annealing and extension.

The polymerase can be added to the random polynucleotides prior to annealing, simultaneously with annealing or after annealing.

The cycle of denaturation, annealing and incubation in the presence of polymerase is referred to herein as shuffling or reassembly of the nucleic acid. This cycle is repeated for a desired number of times. Preferably the cycle is repeated from 2 to 50 times, more preferably the sequence is repeated from 10 to 40 times. However, it is understood that any suitable number of repeated cycles can be performed so long as the sesired product of the reduction is achieved.

The resulting nucleic acid is a larger double-stranded polynucleotide of from about 50 bp to about 100 kb, preferably the larger polynucleotide is from 500 bp to 50 kb. These polynucleotides are then cloned into the appropriate vector and the ligation mixture used to transform bacteria.

The vector used for cloning is not critical provided that it will accept a polynucleotide of the desired size. If expression of the particular polynucleotide is desired, the cloning vehicle should further comprise transcription and translation signals next to the site of insertion of the polynucleotide to allow expression of the polynucleotide in the host cell. Preferred vectors include the pUC series and the pBR series of plasmids.

The resulting bacterial population will include a number of recombinant polynucleotides having random mutations. This mixed population can be tested to identify the desired recombinant polynucleotides. The method of testing will depend on the polynucleotide desired.

For example, if a polynucleotide which encodes a protein with increased binding efficiency to a ligand is desired, the proteins expressed by each of the portions of the polynucleotides in the population or library can be tested for their ability to bind to the ligand by methods known in the art (i.e., panning, affinity chromatography). If a polynucleotide which encodes a protein with increased drug resistance is desired, the proteins expressed by each of the polynucleotides in the population or library can be tested for their ability to confer drug resistance to the host organism. One skilled in the art, given knowledge of the desired protein, can readily test the population to identify polynucleotides which confer the desired properties onto the protein.

It is contemplated that one skilled in the art can use a phage display system in which fragments of the protein are expressed as fusion proteins on the phage surface. The recombinant DNA molecules are cloned into the phage DNA at a site which results in the transcription of a fusion protein a portion of which is encoded by the recombinant DNA molecule. The phage containing the recombinant nucleic acid molecule undergoes replication and transcription in the cell. The leader sequence of the fusion protein directs the transport of the fusion protein to the tip of the phage particle. Thus, the fusion protein, which is partially encoded by the recombinant DNA molecule, is displayed on the phage particle for detection and selection by the methods described above.

It is further contemplated that a number of cycles of nucleic acid shuffling can be conducted with polynucleotides from a sub-population of the first population, which sub-population contains DNA encoding the desired recombinant protein. In this manner, proteins with even higher binding affinities or enzymatic activity or other describable properties can be achieved.

It is also contemplated that a number of cycles of nucleic acid shuffling can be conducted with a mixture of wild-type polynucleotides and a sub-population of nucleic acid from the first or subsequent rounds of nucleic acid shuffling in order to remove any silent mutations from the sub-population.

Any source of nucleic acid, preferably in purified form, can be utilized as the starting nucleic acid. Thus the process can employ DNA or RNA, including messenger RNA (mRNA). The nucleic acid sequence can be of various lengths depending on the size of the nucleic acid sequence to be mutated. Preferably the specific nucleic acid sequence is from 50 to 50,000 nucleotides. It is contemplated that entire vectors containing the nucleic acid encoding the protein of interest can be used in the methods of the invention.

The nucleic acid can be obtained from any source, for example, from plasmids such as pBR322, from cloned DNA or RNA or from natural DNA or RNA from any source including bacteria, yeast, viruses and higher organisms such as plants or animals. DNA or RNA can be extracted from blood or tissue material. The template polynucleotide can be obtained by amplification using the polynucleotide chain reaction (PCR) (U.S. Pat. Nos. 4,683,202 and 4,683,195). Alternatively, the polynucleotide can be present in a vector present in a cell and sufficient nucleic acid can be obtained by culturing the cell and extracting the nucleic acid from the cell by methods known in the art.

Any specific nucleic acid sequence can be used to produce the population of mutants by the present process. It is only necessary that a small population of mutant sequences of the specific nucleic acid sequence exist or be created prior to the present process.

The initial small population of the specific nucleic acid sequences having mutations can be created by a number of different methods. Mutations can be created by error-prone PCR. Error-prone PCR uses low-fidelity polymerization conditions to introduce a low level of point mutations randomly over a long sequence. Alternatively, mutations can be introduced into the template polynucleotide by oligonucleotide-directed mutagenesis. In oligonucleotide-directed mutagenesis, a short sequence of the polynucleotide is removed from the polynucleotide using restriction enzyme digestion and is replaced with a synthetic polynucleotide in which various bases have been altered from the original sequence. The polynucleotide sequence can also be altered by chemical mutagenesis. Chemical mutagens include, for example, sodium bisulfite, nitrous acid, hydroxylamine, hydrazine or formic acid. Other agents which are analogues of nucleotide precursors include nitrosoguanidine, 5-bromouracil, 2-aminopurine, or acridine. Generally, these agents are added to the PCR reaction in place of the nucleotide precursor thereby mutating the sequence. Intercalating agents such as proflavine, acriflavine, quinacrine and the like can also be used. Random mutagenesis of the polynucleotide sequence can also be achieved by irradiation with X-rays or ultraviolet light. Generally, plasmid polynucleotides so mutagenized are introduced into a suitable host such as E. coli and propagated as a pool or library of mutant plasmids.

Alternatively the small mixed population of specific nucleic acids can be found in nature in that they can consist of different alleles of the same gene or the same gene from related species (i.e., cognate genes). Alternatively, they can be related DNA sequences found within one species, for example, the immunoglobulin genes.

Once the mixed population of the specific nucleic acid sequences is generated, the polynucleotides can be used directly or inserted into an appropriate cloning vector, using techniques well-known in the art. The choice of vector depends on the size of the polynucleotide sequence and the host cell to be employed in the methods of the invention. The templates used in methods of invention can be derived from plasmids, phages, cosmids, phagemids, viruses (e.g., retroviruses, parainfluenzavirus, herpesviruses, reoviruses, paramyxoviruses, and the like), or selected portions thereof (e.g., movement protein, coat protein, spike glycoprotein, capsid protein). For example, cosmids and phagemids are preferred where the specific nucleic acid sequence to be mutated is larger because these vectors are able to stably propagate large polynucleotides.

If the mixed population of the specific nucleic acid sequence is cloned into a vector, it can be clonally amplified by inserting each vector into a host cell and allowing the host cell to amplify the vector. This is referred to as clonal amplification because, while the absolute number of nucleic acid sequences increases, the number of mutants does not increase. Useful mutants can be readily determined by testing expressed polypeptides.

The DNA shuffling method of the invention can be performed blindly on a pool of unknown sequences. By adding to the reassembly mixture oligonucleotides with ends that are homologous to the sequences being reassembled, any sequence mixture can be incorporated at any specific position into another sequence mixture. Thus, it is contemplated that mixtures of synthetic oligonucleotides, PCR polynucleotides or even whole genes can be mixed into another sequence library at defined positions. The insertion of one sequence (mixture) is independent from the insertion of a sequence in another part of the template. Thus, the degree of recombination, the homology required, and the diversity of the library can be independently and simultaneously varied along the length of the reassembled DNA.

This approach of mixing two genes can be useful for the humanization of antibodies from murine hybridomas. The approach of mixing two genes or inserting mutant sequences into genes can be useful for any therapeutically useful protein, for example, interleukin I, antibodies, tPA, growth hormone, and the like. The approach can also be useful in any nucleic acid, for example, promoters or introns or 3′ untranslated region or 5′ untranslated regions of genes to increase expression or alter specificity of expression of proteins. The approach can also be used to mutate ribozymes or aptamers.

Shuffling requires the presence of homologous regions separating regions of diversity. Scaffold-like protein structures can be particularly suitable for shuffling. The conserved scaffold determines the overall folding by self-association, while displaying relatively unrestricted loops that mediate the specific binding. Examples of such scaffolds are the immunoglobulin-barrel, and the four-helix bundle which are well-known in the art. Shuffling can be used to create scaffold-like proteins with various combinations of mutated sequences for binding.

### In Vitro Shuffling

The equivalents of some standard genetic matings can also be performed by shuffling in vitro. For example, a “molecular backcross” can be performed by repeatedly mixing the mutant's nucleic acid with the wild-type nucleic acid while screening or selecting for the mutations of interest. As in traditional breeding, this approach can be used to combine phenotypes from different sources into a background of choice. It is useful, for example, for the removal of neutral mutations that affect unselected characteristics (i.e. immunogenicity). Thus, it can be useful to determine which mutations in a protein are involved in the enhanced biological activity and which are not, an advantage which cannot be achieved by error-prone mutagenesis or cassette mutagenesis methods.

Large, functional genes can be assembled correctly from a mixture of small random polynucleotides. This reaction can be of use for the reassembly of genes from the highly fragmented DNA of fossils. In addition, random nucleic acid fragments from fossils can be combined with polynucleotides from similar genes from related species.

The polynucleotide to be shuffled can be fragmented randomly or non-randomly, as desired, by the methods disclosed herein.

In general, standard techniques of recombination DNA technology are described in various publications, e.g. Sambrook et al., Molecular Cloning: A Laboratory Manual, Cold Spring Harbor Laboratory (1989); Ausubel et al., 1999, Current Protocols in Molecular Biology, and Kimmel and Berger, Methods Enzymol. Vol. 152. Guide to Molecular Cloning Techniques (1987), Academic Press, Inc., San Diego, Calif., each of which is incorporated herein in their entirety by reference. Polynucleotide modifying enzymes are generally used according to the manufacturers recommendations. If desired, PCR amplimers for amplifying a predetermined DNA sequence can be chosen at the discretion of the practitioner.

The following non-limiting examples are provided to illustrate the present invention.

### EXAMPLE 1

### DNA Shuffling of Green Fluorescent Protein

This example describes DNA shuffling of two frameshifted, non-fluorescent green fluorescent protein (gfp) genes to reconstitute active gfp.

Two frameshifted and non-fluorescent gfp genes were used. One gfp gene was frameshifted at the 5′ end (designated 5′ FS) by changing the first ATG to GGCC by oligonucleotide-directed mutagenesis, and the other was frameshifted near the 3′ end (designated 3′ FS) by digesting with SalI to linearize the plasmid at nucleotide position 605 of the gfp open reading frame, filling in the recessed ends with the Klenow fragment of DNA Polymerase I in the presence of all four dNTPs, followed by ligation to re-close the plasmid resulting in a four basepair insertion. The genes were shuffled together and assayed for fluorescence to show recombination between the two non-functional genes to generate functional genes.

To generate single-stranded DNA, PCR products encoding the mutant gfp genes, along with flanking viral sequences of 295 bp at the 5′ end and 176 bp of viral sequences at the 3′ end of the viral vectors, 5′ FS and 3′ FS were first synthesized. The primers hsPCR2-F (ACA GAA GAA GTC GTT GAT GAG TTC; SEQ ID NO: 5) and hsPCR1-R (GCT TTA TTA CGT GCC TGC GGA TG; SEQ ID NO: 6) were used to amplify these products. For making 3′ FS single-stranded DNA, the PCR product encoding the mutant gene was subjected to asymmetric PCR using only a single primer in the cycling reaction (GFP-shuffAmp-F, ATG TTA AGG ATT TTG GAG GAA TGA G; SEQ ID NO: 7). The result was a mixture of double-stranded and single-stranded (top strand) DNA, which was then resolved on a 1.2% agarose gel from which the band containing the single-stranded DNA was excised and then purified using a DNA extraction spin-column (Qiaquick Gel Extraction Kit, QIAGEN, Inc.). For making the 5′ FS single-stranded DNA (bottom strand), the reverse primer (GFP-shuffAmp-R, CGT TAT TTC CTA GGC ATC TTG ACT ACC CCT CGA GTG; SEQ ID NO: 8) was used in the asymmetric PCR reaction, and the single-stranded DNA was purified in a manner similar to the 3′ FS sample. Separately, both single-stranded DNAs were digested with an appropriate amount of DNAseI, and the resulting fragments were run out on a 3% agarose gel. The smear of DNA ranging from ˜50-500 bases was excised and processed by DNA spin-column to obtain random fragments of single-stranded DNA.

For the reassembly reaction, individual single-stranded DNA samples derived from 3′ FS and 5′ FS were mixed and added to a reaction containing a mixture of Taq and Pfu polymerases with dNTPS and the appropriate buffer (Barnes, W. M. (1994) Proc. Natl. Acad. Sci USA, 91, 2216-2220) and cycled in a thermocycler 94° C., 30 sec, (94° C.-5 sec, 55° C.-30 sec, 72° C.-1 min)×15 cycles. An aliquot was run on a gel and showed a marked increase in average size in the smear.

For amplification of full-length reassembled recombinants, 1 μl of the reassembly reaction was added to a PCR mixture (similar to above) along with primers that were used to generate the single-stranded DNAs and cycled in a thermocycler 94° C., 30 sec, (94° C.-5 sec, 45° C.-30 sec, 72° C.-2 min)×15 cycles. An aliquot was run on a gel and the visible amplification product was of the expected size. The DNA band was excised and processed to isolate the DNA, which was then digested with PacI and AvrII and inserted between the PacI and AvrII sites of the viral vector, p5.5XPL.

Of 24 clones that were isolated, 23 showed full-length viral vector constructs with an insertion of the gfp shuffling product. The 23 full-length DNA preparations were used for in vitro transcription along with appropriate positive and negative controls. Of the 23 that were inoculated onto a local-lesion tobacco host, 9 were fluorescent. The shuffled clones are expected to include double-frameshifts, single-frameshifts (recombination upstream of 5′FS or downstream of 3′FS ), or wild-type (that is, loss of both frameshifts). Therefore, the observed 9 of 23 clones having fluorescent activity indicated that recombination at points between the 5′ and 3′ frameshift sites occurred at a reasonable frequency. DNA sequencing is performed to determine the proportions of possible shuffled clones that are inactive (i.e., double-frameshift or single-frameshifts).

This example demonstrates highly efficient DNA shuffling to generate wild type gfp encoding active GFP from frameshifted parental molecules encoding inactive GFP.

### EXAMPLE 2

### Molecular Breeding of the 30K Movement Proteins of Tobamoviruses I

This example describes DNA shuffling of two related but significantly divergent genes encoding the 30K movement protein of tobamoviruses, a process which is known as molecular breeding.

Movement protein (MP) genes from two species of tobamoviruses were used. Tobacco mosaic virus (TMV) and Tomato mosaic virus (ToMV) were chosen because they are only ˜75% identical at the nucleotide level (Oliver, et al., Virology 155(1986) 277-283; Weber et al., J Virol 66(1992)3909-3912; Matsushita et al., J Gen Virol 81(2000)2095-2102). Differences between the two genes (TMV, UIMP; ToMV, ToMVMP) are distributed rather evenly throughout the sequences and tend to be concentrated in wobble positions. Thus, at the amino acid level, these proteins are more highly related, increasing the likelihood that recombinants will retain function.

The goals of this experiment were: (1) to establish whether all products of the shuffling reaction were recombinants and were full-length genes, with crossovers occurring at homologous positions within the genes; (2) to examine the shufflants at the sequence level to determine whether crossover junctions were precise and conservative of reading frame and free of point mutations and frameshifts; and (3) to assay each resulting shufflant for function, both for local cell-to-cell and for subgenomic promoter activity.

The protocols used for MP heterosexual PCR (hsPCR) were similar to those used for the GFP mutants (Example 1). One major difference between this experiment and how the GFP experiment was performed was the use of clamps. Terminal sequence “clamps” of about 80 bp were added to the MP gene ssDNAs prior to fragmentation and reassembly. This was done to facilitate recovery of small, terminal fragments of ssDNA. Perhaps more significantly, the clamps were also used to further ensure that only recombinant molecules would be recovered after reassembly and amplification. Briefly, one of the MP genes was given clamp “A” at its 51′ end, and the other MP gene was given clamp “B” at its 3′ end. After fragmentation and reassembly of the single stranded DNAs encoding these singly-clamped MP genes, amplification was performed using clamp A- and clamp B-specific primers. Thus, only molecules bearing clamps at both ends (i.e., recombinants) will be amplified. This provides further assurance that non-recombined parental molecules will not be recovered after shuffling.

For production of clamped ssDNAs, each MP was amplified with gene-specific primers that annealed to the termini of the open reading frames (ORFs) of the MP genes. Primer sequences were as follows:

| | |
|---|---|
| U1MP5′, GGC ACA AGT TAC AAG CAG TCA CGC AAC CCT AGG ATG GCT CTA GTT GTT AAA GGA AAA G | (SEQ ID NO:9); |
| U1MP3′, ACT ACC CTG TAA ATA GAC ACT TAC TGC AGT TAA TTA AAA CGA ATC CGA TTC GGC GAC | (SEQ ID NO:10); |
| ToMP5′, GGC ACA AGT TAC AAG CAG TCA CGC AAC CCT AGG ATG GCT CTA GTT GTT AAA GGT AAG G | (SEQ ID NO:11); |
| ToMP3′, ACT ACC CTG TAA ATA GAC ACT TAC TGC AGT TAA TTA ATA CGA ATC AGA ATC CGC GAC | (SEQ ID NO:12). |

These primers were each flanked by unique restriction sites, AvrII for 5′ primers and PacI for 3′ primers, which were, in turn, flanked by additional non-coding spacer sequence. To generate the clamped single-strands, the PCR products were used as templates in asymmetric PCR reactions that included only a single primer in the cycling reaction. For the TMV reaction, the primer (sense-direction) that was used (5′ MPss, GTA CGT AGT CAC GAC ATT AGA GTA ACA CGC GTG AGG CAC AAG TTA CAA GCA GTC ACG C; SEQ ID NO: 13) annealed to the 5′ end of the spacer that flanked the 5′ end of the MP gene. This primer contained 34 bases of unique 5′ flanking sequences called “clamp A” (clamp A-specific primer). For the ToMV reaction, the primer (reverse-sense) that was used (3′MPss, GAA TAG AAC ACT CAT TGA TAC GGA TTT ATA CAT GAC TAC CCT GTA AAT AGA CAC TTA C; SEQ ID NO: 14) annealed to the 3′ end of the spacer that flanked the 3′ end of the MP gene. This primer contained 34 bases of unique 3′ flanking sequences called “clamp B” (clamp B-specific primer). The results of these PCR reactions were mixtures of double-stranded and single-stranded DNAs, which were then resolved on a 1.2% agarose gel from which the bands containing the single-stranded DNA were excised and processed using DNA extraction spin-columns. Separately, both single-stranded DNAs were fragmented with an appropriate amount of DNAseI, and the resulting fragments were separated on a 3% agarose gel. The smear of DNA ranging from ˜50-500 bases was excised and processed by DNA spin-columns to obtain random fragments of single stranded DNA.

For the reassembly reaction, individual ssDNA samples derived from TMV and ToMV were mixed and added to a reaction containing a mixture of Taq and Pfu polymerases with DNTPS and the appropriate buffer (Barnes, W. M. supra(1994)) and cycled in a thermocycler 94° C., 30 sec, (94° C.-5 sec, 30° C.-1 min, 72° C.-1 min)×20 cycles.

For amplification of full-length reassembled recombinants, 1 μl of the reassembly reaction was added to a PCR mixture (similar to Example 1), along with a clamp A specific primer (5′MPamp, GTA CGT AGT CAC GAC ATT AGA G; SEQ ID NO:15) and a clamp B specific primer (3′MPamp, GAA TAG AAC ACT CAT TGA TAC GG; SEQ ID NO:16) and cycled in a thermocycler 94° C., 30 sec, (94° C.-5 sec, 45° C.-15 sec, 72° C.-1.5 min)×20 cycles. An aliquot was run on a gel and the visible amplification product was of the expected size. The DNA band was excised and processed to isolate the DNA, which was then digested with the AvrII and PacI and inserted into a similarly-cut p30B- MP viral vector.

Of 24 clones that were purified as miniprep DNA, 16 showed full-length viral vector, with an insertion of the MP shuffling product as indicated by digestion with AvrII and PacI followed by gel fractionation.

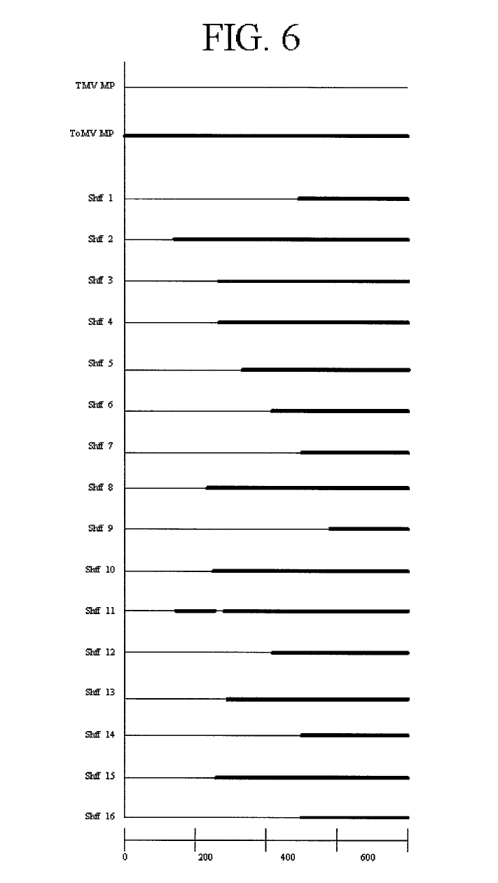

The inserts of the 16 full-length viral vector clones were sequenced. It was determined that all were TMV/ToMV recombinants (15 single crossover events and one clone with multiple crossovers). All recombinations occurred at homologous regions that conserved overall gene structure, and none of the junction sequences were mutated or altered the reading frame. Consistent with reports in the literature, point mutations were found outside of the crossover regions at a rate of several per gene, probably resulting from the inherent mutagenicity of PCR. It was noted that each of the recombinants was more similar to TMV at the 5′ end, and more similar to ToMV at the 3′ end (see FIG. 6). The sequences of these clones, and the wild-type viral MP gene sequences are shown in Table 1. Table 1. Sequence of movement protein genes for TMV and ToMV and shuffled TMV/ToMVMP recombinants:

U1MP (SEQ ID NO:17)
ATGGCTCTAGTTGTTAAAGGAAAAGTGAATATCAATGAGTTTATCGACCTGACAAAAATGGAGAAGATCTTACCGTCGAT
GTTTACCCCTGTAAAGAGTGTTATGTGTTCCAAAGTTGATAAAATAATGGTTCATGAGAATGAGTCATTGTCAGAGGTGA
ACCTTCTTAAAGGAGTTAAGCTTATTGATAGTGGATACGTCTGTTTAGCCGGTTTGGTCGTCACGGGCGAGTGGAACTTG
CCTGACAATTGCAGAGGAGGTGTGAGCGTGTGTCTGGTGGACAAAAGGATGGAAAGAGCCGACGAGGCCACTCTCGGATC
TTACTACACAGCAGCTGCAAAGAAAAGATTTCAGTTCAAGGTCGTTCCCAATTATGCTATAACCACCCAGGACGCGATGA
AAAACGTCTGGCAAGTTTTAGTTAATATTAGAAATGTGAAGATGTCAGCGGGTTTCTGTCCGCTTTCTCTGGAGTTTGTG
TCGGTGTGTATTGTTTATAGAAATAATATAAAATTAGGTTTGAGAGAGAAGATTACAAACGTGAGAGACGGAGGGCCCAT
GGAACTTACAGAAGAAGTCGTTGATGAGTTCATGGAAGATGTCCCTATGTCGATCAGGCTTGCAAAGTTTCGATCTCGAA
CCGGAAAAAAGAGTGATGTCCGCAAAGGGAAAAATAGTAGTAATGATCGGTCAGTGCCGGAACAAGAACTATAGAAATGTT
AAGGATTTTGGAGGAATGAGTTTTAAAAAGAATAATTTAATCGATGATGATTCGGAGGCTACTGTCGCCGAATCGGATTCGTTTTAA
ToMVMP (SEQ ID NO:18)
ATGGCTCTAGTTGTTAAAGGTAAGGTAAATATTAATGAGTTTATCGATCTGTCAAAGTCTGAGAAACTTCTCCCGTCGAT
GTTCACGCCTGTAAAGAGTGTTATGGTTTCAAAGGTTGATAAGATTATGGTCCATGAAAATGAATCATTGTCTGAAGTAA
ATCTCTTAAAAGGTGTAAAACTTATAGAAGGTGGGTATGTTTGCTTAGTCGGTCTTGTTGTGTCCGGTGAGTGGAATTTA
CCAGATAATTGCCGTGGTGGTGTGAGTGTCTGCATGGTTGACAAGAGAATGGAAAGAGCGGACGAAGCCACACTGGGGTC
ATATTACACTGCTGCTGCTAAAAAGCGGTTTCAGTTTAAAGTGGTCCCAAATTACGGTATTACAACAAAGGATGCAGAAA
AGAACATATGGCAGGTCTTAGTAAATATTAAAAATGTAAAAATGAGTGCGGGCTACTGCCCTTTGTCATTAGAATTTGTG
TCTGTGTGTATTGTTTATAAAAATAATATAAAATTGGGTTTGAGGGAGAAAGTAACGAGTGTGAACGATGGAGGACCCAT
GGAACTTTCGGAAGAAGTTGTTGATGAGTTCATGGAGAATGTTCCAATGTCGGTTAGACTCGCAAAGTTTCGAACCAAAT
CCTCAAAAAGAGGTCCGAAAAATAATAATAATTTAGGTAAGGGGCGTTCAGGCGGAAGGCCTAAACCAAAAAGTTTTGAT
GAAGTTGAAAAAGAGTTTGATAATTTGATTGAAGATGAAGCCGAGACGTCGGTCGCGGATTCTGATTCGTATTAA
MPshf1 (SEQ ID NO:19)
CCTAGGATGCCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGCGTGTGTCTGGTGGACAAAAGGATGGAAAGAGCCGACGAGGCCATTCT
CGGATCTTACTACACAGCAGCTGCAAAGAAAAGATTTCAGTTCAAGGTCGTTCCCAATTATGCTATAACCACCCAGGACG
CGATGAGAAACGTCTGGCAAGTTTTAGTTAATATTAGAAATGTGAAGATGTCAGCGGGTTTCTGTCCGCTTTCTCTGGAG
TTTGTGTCGGTGTGTATTGTTTATAAAAATAATATAAAATTGGGTTTGAGGGAGAAAGTAGCGAGTGTGAACGATGGAGG
ACCCATGGAACTTTCAGAAGAAGTTGTTGATGAGTTCATGGAGAATGTTCCAATGTCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGaaAAAGAGTTTGATAATTTGATTGAAGATGAAGCCGAGACGTCGGTCGCAGATTCTGATTCGTATTAATTAA
MPshf 2 (SEQ ID NO:20)
CCTAGGATGGCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCGAAGTTGATAAAATAATGGTCCATGAAAATGAATCATTGTCTG
AAGTAAATCTCTTAAAAGGTGTAAAACTTATAGAAGGTGGGTATGTTTGCTTAGTTGGTCTTGTTGTGTCCGGTGAGTGG
AATTTACCAGATAATTGCCGTGGTGGTGTGAGTGTCTGCATGGTTGACAAGAGAATGGAAAGAGCGGACGAAGCCACACT
GGTGTCATATTACACTGCTGCTGCTAAAAAGCGGTTTCAGTTTAAAGTGGTCCCAAATTACGGTATTACTACAAAGGATG
CAGAAAAGAACATAAGGCAGGTCTTAGTAAATATTAAAAATGTAAAAATGAGTGCGGGCTACTGCCCTTTGTCATTAGAA
TTTGTGTCTGTGTGTATTGTTTATAAAAATAATATAAAAATTGGGTTTGAGGGAGAAAGTAACGAGTGTGAACGATGGAGG
ACCCATGGAACTTTCAGAAGAAGTTGTTGATGAGTTCATGGAGAATGTTCCAATGTCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGaAAAAGAGTTTGATAATTTGATTGAAGATGAAGCCGAGACGTCGGTCGCGGATTCTGATTCGTATTAATTAA
MPshf 3 (SEQ ID NO:21)
CCTAGGATGGCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGTGTCTGCATGGTTGACAAGAGAATGGAAAGAGCGGACGAAGCCACACT
GGGGTCATATTACACTGCTGCTGCTAAAAAGCGGTTTCAGTTTAAAGTGGTCCCAAATTACGGTATTACTACAAAGGATG
CAGAAAAGAACATATGGCAGGTCTTAGTAAATATTAAAAATGTAAAAATGAGTGCGGGCTACTGCCCTTTGTCATTAGAA
TTTGTGTCTGTGTGTATTGTTTATAAAAATAATATAAAATTGGGTTTGAGGGAGAAAGTAACGAGTGTGAACGATGGAGG
ACCCATGGAACTTTCAGAAGAAGTTGTTGATGAGTTCATGGAGAATGCTCCAATGTCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGAAAAAGAGTTTGATAATTTGATTGAAGATGAAGCCGAGACGTCGGTCGCGGATTCTGATTCGTATTAATTAA
MPshf 4 (SEQ ID NO:22)
CCTAGGATGGCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGTGTCTGCATGGTTGACAAGAGAATGGAAAGAGCGGACGAAGCCACACT
GGGGTCATATTACACTGCTGCTGCTAAAAAGCGGTTTCAGTTTAAAGTGGTCCCAAATTACGGTATTACTACAAAGGATG
CAGAAAAGAACATATGGCAGGTCTTAGTAAATATTAAAAATGTAAAAATGAGTGCGGGCTACTGCCCTTTGTCATTAGAA
TTTGTGTCTGTGTGTATTGTTTATAAAAATAATATAAAATTGGGTTTGAGGGAGAAAGTAACGAGTGTGAACGATGGAGG
ACCAATGGAACTTTCAGAAGAAGTTGTTGATGAGTTCATGGAGAATGTTCCAATATCGGTTAGACTCGCAAAGTTTCGAA
CCAAATCCTCAAAAAGAGGTACGAAAAAATAATAATAATTTAGGTAAGGGGCGTTCAGGCGGAAGGCCTAAACCAAAAAG
TTTTGATGAAGTTGAAAAAGAGTTTGATAATTTGATTGAAGATGAAGCCGAGACGTCGGTCGCGGATTCTGATTCGTATTAATTAA
MPshf 5 (SEQ ID NO:23)
CCTAGGATGGCTCTAGTTGTTAAAGGAAAAGTGAATATCAATGAGTTTATCGACCTGACAAAAATAGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGCGTGTGTCTGGTGGACAAAAGGATGGAAAGAGCCGACGAGGCCATTCT
CGGATCTTACTACACTGCTGCTGCTAAAAAGCGGTTTCAGTTTAAAGTGGTCCCAAATTACGGTATTACTACAAAGGATG
CAGAAAAGAACATATGGCAGGTCTTAGTAAATATTAAAAATGTAAAAATGAGTGCGGGCTACTGCCCTTTGTCATTAGAA
TTTGTGTCTGTGTGTATTGTTTATAAAAATGATATAAAATTGGGTTTGAGGGAGAAAGTAACGAGTGTGAACGATGGAGG
ACCCATGGAACTTTCAGAAGAAGTTGTTGATGAGTTCATGGAGAATGTTCCAATGTCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGAAAAAGAGTTTGATAATTTGATTGAAGATGAAGCCGAGACGTCGGTCGCGGATTCTGATTCGTATTAATTAA
MPshf 6 (SEQ ID NO:24)
CCTAGGATGGCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAGTGAGTCATTGTCAG
GGGTGAACCTTCTTAAAGGAGTTAAGCTTATTGATAGTGGATACGTCCGTTTAGCCGGTTTGGTCGTCACGGGCGAGTGG
AACTTGCCTGACAATTGCAGAGGAGGTGTGAGCGTGTGTCTGGTGGACAAAAGGATGGAAAGAGCCGACGAGGCCATTCT
CGGATCTTACTACACAGCAGCTGCAAAGAAAAGATTTCAGTTCAAGGTCGTTCCCAATTATGCTATAACCACCCAGGACG
CGATGAGAAACGTCTGGCAAGTTTTAGTAAATATTAAAAATGTAAAAATGAGTGCGGGCTACTGCCCTTTGTCATTAGAA
TTTGTGTCTGTGTGTATTGTTTATAAAAATAATATAAAATTGGGTTTGAGGGAGAAAGTAACGAGTGTGAACGATGGAGG
ACATGGAACTTTCAGAAGAAGTTGTTGATGAGTTCATGGAGAATGTTCCAATGTCGGTTAGACTCGCAAAGTTTCGAACC
AAATCCTCAAAAAGAGGTCCGAAAAATAATAATAATTTGGTAAGGGGCGTTCAGGCGGAAGGCCTAAACCAAAAAGTTTT
GATGAAGTTGAAAAAGAGTTTGATAATTTGATTGAAGATGAAGCCGAGACGTCGGTCGCGGATTCTGATTCGTATTAATTAA
MPshf 7 (SEQ ID NO:25)
CCTAGGATGGCTCTAGTTGTTAAAGGAAAAGTGAATATCAATGAGTTTATCGACCTGACAAAAATGGAGAAGATCCTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTTGCCGGTTTGGTCGTCACGGGCGAGTGG
AACTTGCCTGACAATTGCAGAGGAGGTGTGAGCGTGTGTCTGGTGGACAAAAGGATAGAAAGAGCCGACGAGGCCATTCT
CGGATCTTACTACACAGCAGCTGCAAAGAAAAGATTTCAGTTCAAGGTCGTTCCCAATTATGCTATAACCACCCAGGACG
CGATGAGAAACGTCTGGCAAGTTTTAGCTAATATTAGAAATGTGAAGATGTCAGCGGGTTTCTGTCCGCTTTCTCTGGAG
TTTGTGTCGGTGTGTATTGTTTATAAAAATAATATAAAATTGGGTCTGAGGGAGAAAGTAACGAGTGTGAACGATGGAGG
ACCCATGGAACTTTCAGAAGAAGTTGTTGATGAGTTCATGGAGAATGTTCCAATGTCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGCCAAAGAGTTTGATAATTTGATTGAAGATGAAGCCGAGACGTCGGTTGCGGATTCTGATTCGTATTAATTAA
MPshf 8 (SEQ ID NO:26)
CCTAGGATGGCTCTCGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AATTTACCAGATAATTGCCGTGGTGGTGTGAGTGTCTGCATGGTTGACAAGAGAATGGAAAGAGCGGACGAAGCCACACT
GGGGTCATATTACACTGCTGCTGCTAAAAAGCGGTTTCAGTTTAAAGTGGTCCCAAATTACGGTACTACTACAAAGGATG
CAGAAAAGAACATATGGCAGGTCTTAGTAAATATTAAAAATGTAAAAATGAGTGCGGGCTACTGCCCTTTGTCATTAGAA
TTTGTGTCTGTGTGTATTGTTTATAAAAATAATATAAAATTGGGTTTGAGGGAGAAAGTAACGAGTGTGAACGATGGAGG
ACCCATGGAACTTTCAGAAGAAGTTGTTGATGAGTTCATGGAGAATGTTCCAATGTCGGTTAGGCTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGAAAAAGAGTTTGATAATTTGATTGAAGATGAAGCCGAGACGTCGGTCGCGGATTCTGATTCGTATTAATTAA
MPshf 9 (SEQ ID NO:27)
CCTAGGATGTCTCTAGTTGTTAAAGGAAAAGTGAATATCAATGAGTTTATCGACCTGACAAAAATGGAGAAGATCTTACC
GTCGATGTTTACCCCTGTAAGGAGTGTTATGTGTTCCAAAGTTGATAAAACAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGCGTGTGTCTGGTGGACAAAAGGATGGAAAGAGCCGACGAGGCCATTCT
CGGATCTTACTACACAGCAGCTGCAAAGAAAAGATTTCAGTTCAAGGTCGTTCCCAATTATGCTATAACCACCCAGGACG
CGATGAGAAACGTCTGGCAAGTTTTAGTTAATATTAGAAATGTGAAGATGTCAGCGGATTTCTGTCCGCTTTCTCTGGAG
TTTGTGTCGGTGTGTATTGTTTATAGAAATAATATAAAATTAGGTTTGAGAGAGAAGATTACAAACGTGAGAGACGGAGG
GCCCATGGAACTTTCAGAAGAAGTTGTTGATGAGTTCATGGAGAATGTTCCAAAGTCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGGAGTTGAAAAAGAGTTTGATAATTTGATTGAAGATGAAGCCGAGACGTCGGTCGCGGATTCTGATTCGTATTAATTAA
MPshf 10 (SEQ ID NO:28)
CCTAGGATGGCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCAGATAATTGCCGTGGTGGTGTGAGTGTCTGCATGGTTGACAAGAGAATGGAAAGAGCGGACGAAGCCACACT
GGGGTCATATTACACTGCTGCTGCTAAAAAGCGGTTTCAGTTTAAAGTGGTCCCAAATTACGGTATTACTACAAAGGATG
CAGAAAAGAACATATGGCAGGTCTTAGTAAATATTAAAAATGTAAAAATGAGTGCGGGCTACTGCCCTTTGTCATTAGAA
TTTGTGTCTGTGTGTATTGTTTATAAAAATAATATAAAATTGGGTTTGAGGGAGAAAGTAACGAGTGTGAACGATGGAGG
ACCATGGAACTTTCAGAAGAAGTTGTTGATGAGTTCATGGAGAATGTTCCAATGTCGGTTAGACTCGCAAAGTTTCGAAC
CAAATCCTCAAAAAGAGGTCCGAAAAATAATAATAATTTAGGTAAGGGGCGTTCAGGCGGAAGGCCTAAACCAAAAAGTT
TTGATGAAGTTGAAAAAGAGTTTGATAATTTGATTGAAGATGGAGCCGAGACGTCGGTCGCGGATTCTGATTCGTATTAATTAA
MPshf 11 (SEQ ID NO:29)
CCTAGGATGCCTCTAGTTGTTAAAGGAAAAGTGAATATCAATGAGTTTACCGACCTGACAAAAATGGAGAAGATCTTACC
GTCGATGTTTACCCCTGTAAAGAGTGTTATGTGTTCCAAAGTTGATAAAATAATGGTTCATGAAAATGAATCATTGTCTG
AAGTAAATCTCTTAAAAGGTGTAAAACTTATAGAAGGTGGGTATGTTTGCTTAGTTGGTCTTGTTGTGTCCGGTGAGTGG
AATTTACAGATAATTGCAGAGGAGGTGTGAGCGTGTGTCTGGTGGACAAAAGGATGGAAAGAGCGGACGAAGCCACACTG
GGGTCATATTGCACTGCTGCTGCTAAAAAGCGGTTTCAGTTTAAAGTGGTCCCAAATTACGGTATTACTACAAAGGATGC
AGAAAAGAACATATGGCAGGTCTTAGTAAATATTAAAAATGTAAAAATGAGTGCGGGCTACTGCCCTTTGTCATTAGAAT
TTGTGTCTGTGTGTATTGTTTATAAAAATAATATAAAATTGGGTTTGAGGGAGAAAGTAACGAGTGTGAACGATGGAGGA
CCCATGGAACTTTCAGAAGAAGTTGTTGATGAGTTCATGGAGAATGTTCCAATGTCGGTTAGACTCGCAAAGTTTCGAAC
CAAATCCTCAAAAAGAGGTCCGAAAAATAATAATAATTTAGGTAAGGGGCGTTCAGGCGGAAGGCCTAAACCAAAAAGTT
TTGATGAAGTTGAAAAAGAGTTTGATAATTTGATTGAAGATGAAGCCGAGGCGTCGGTCGCGGATTCTGATTCGTATTAATTAA
MPshf 12 (SEQ ID NO:30)
CCTAGGATGCCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGCGTGTGTCTGGTGGACAAAAGGATGGAAAGAGCCGACGAGGCCATTCT
CGGATCTTAATACACAGCAGCTGCAAAGAAAAGATTTCAGTTCAAGGTCGTTCCCAATTATGCTATAACCACCCAGGACG
CGAAAAGAACATATGGCAGGTCTTAGTAAATATTAAAAATGTAAAAATGAGTGCGGGCTACTGCCCTTTGTCATTAGAAT
TTGTGTCTGTGTGTATTGTTTATAAAAATAATATAAAATTGGGTTTGAGGGAGAAAGTAACGAGTGTGAACGATGGAGGA
CCCATGGAACTTTCAGAAGAAGTTGTTGATGAGTTCATGGAGAATGTTCCAATGTCGGTTAGACTCGCAAAGTTTCGAGC
CAAATCCTCAAAAAGAGGTCCGAAAAACAATAATAATTTAGGTAAGGGGCGTTCAGGCGGAAGGCCTAAACCAAAAAGTT
TTGATGAAGTTGAAAAAGAGTTTGATAATTTGATTGAAGATGAAGCCGAGACGTCGGTCGCGGATTCTGATTCGTATTAATTAA
MPshf 13 (SEQ ID NO:31)
CCTAGGATGGCTCTAATTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGCGTGTGTCTGGTGGACAAGAGAATGGAAAGAGCGGACGAAGCCACACT
GGGGTCATATTACACTGCTGCTGCTAAAAAGCGGTTTCAGTTTAAAGTGGTCCCAAATTACGGTATTACTACAAAGGATG
CAGAAAAGAACATATGGCAGGTCTTAGTAAATATTAAAAATGTAAAAATGAGTGCGGGCTACTGCCCTTTGTCATTAGAA
TTTGTGTCTGTGTGTATTGTTTATAAAAATAATATAAAATTGGGTTTGAGGGAGAAAGTAACGAGTGTGAACGATGGAGG
ACCCATGGAACTTTCAGAAGAAGTTGTTGATGAGTTCATGGAGAATGTTCCAATGTCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGAAAAAGAGTTTGATAATTTGATTGAAGATGAAGCCGAGACGTCGGTCGCGGATTCTGATTCGTATTAATTAA
MPshf 14 (SEQ ID NO:32)
CCTAGGATGGCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGCGTGTGTCTGGTGGACAAAAGGATGGAAAGAGCCGACGAGGCCATTCT
CGGATCTTACTACACAGCAGCTGCAAAGAAAAGATTTCAGTTCAAGGTCGTTCCCAATTATGCTATAACCACCCAGGACG
CGATGAGAAACGTCTGGCAAGTTTTAGTTAATATTAGAAATGTGAAGATGTCAGCGGGTTTCTGTCCGCTTTCTCTGGAG
TTTGTGTCGGTGTGTATTGTTTATAAAAATAATATAAAATTGGGTTTGAGGGAGAAAGTAACGAGTGTGAACGATGGAGG
ACCCATGGAACTTTCAGAAGAAGTTGTTGATGAGTTCATGGAGAATGTTCCAATGTCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGAAAAAGAGTTTGATAATTTGATTGAAGATGAAGCCGAGACGTCGGTCGCGATTCTGATTCGTATTAATTAA
MPshf 15 (SEQ ID NO:33)
CCTAGGATGGCTCTAGTTGTTAAAGGAAAAGTGAATATCAATGAGTTTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGGTACGTCTGTTTAGCCGGTTTGGTCGTCACGGGCGAGTGG
AACTTGCCTGACAATTGCCGTGGTGGTGTGAGTGTCTGCATGGTTGACAAGAGAATGGAAAGAGCGGACGAAGCCACACT
GGGGTCATATTACACTGCTGCTGCTAAAAAGCGGTTTCAGTTTAAAGTGGTCCCAAATTACGGTATTACTACAAAGGATG
CAGAAAAGAGCATATGGCAGGTCTTAGTAAATATTAAAAATGTAAAAATGAGTGCGGGCTACTGCCCTTTGTCATTAGGA
TTTGTGTCTGTGTGTATTGTTTATAAAAATAATATAAAATTGGGTTTGAGGGAGAAAGTAACGAGTGTGAACGATGGAGG
ACCCATGGAACTTTCAGAAGAAGTTGTTGATGAGTTCATGGAGAATGTTCCAATATCGGTTAGACTCGCAAAGTTTCGAA
CCAAATCCTCAAAAAGAGGTCCGAAAAATAATAATAATTTAGGTAAGGGGCGTTCAGGCGGAAGGCCTAAACCAAAAAGT
TTTGATGAAGTTGAAAAAGAGTTTGATAATTTGATTGAAGATGAAGCCGAGACGTCGGTCGCGGATTCTGATTCGTATTAATTAA
MPshf 16 (SEQ ID NO:34)
CCTAGGATGGCTCTAGTTGTCAAAGGAAAAGTGAATATCAATGAGTCTATCGACCTGACAAAAATGGAGAAGATCTTACC
GTCGATGTTTACCCCTGTAAAGAGTGTTATGTGTTCCAAAGTTGATAAAATAATGGTTCATGAGAATGAGTCATTGTCAG
GGGTGAACCTTCTTAAAGGAGTTAAGCTTATTGATAGTGGATACGTCTGTTTAGCCGGTTTGGTCGTCACGGGCGAGTGG
AACTTGCCTGACAATTGCAGAGGAGGTGTGAGCGTGTGTCTGGTGGACAAAGGATGGAAAGAGCCGACGAGGCCATTCTC
GGATCTTACTACACAGCAGCTGCAAAGAAAAGATTTCAGTTCAAGGTCGTTCCCAATTATGCTATAACCACCCAGGACGC
GATGAGAAACGTCTGGCAAGTTTTAGTTAATATTAGAAATGTGAAGATGTCAGCGGGTTTCTGTCCGCTTTCTCTGGAGT
TTGTGTCGGTGTGTATTGTTTATAAAAATAATATAAAATTGGGTTTGAGGGAGAAAGTAACGAGTGTGAACGATGGAGGG
CCCATGGAACTTTCAGAAGAAGTTGTTGATGAGTTCATGGAGAATGTTCCAATGTCGGTTAGACTCGCAAAGTTTCGAAC
CAAATCCTCAAAAAGAGGTCCGAAAAATAATAATAATTTAGGTAAGGGGCGTTCAGGCGGAAGGCCTAACCAAAAAGTT
TTGATGAAGTTGAAAAAGAGTTTGATAATTTGATTGAAGATGAAGCCGAGACGTCGGTCGCAGATTCTGATTCGTATTAATTAA

The 16 full-length viral vector DNA preps were used for in vitro transcription along with appropriate controls. The subgenomic promoter driving GFP expression in these constructs is imbedded within the MP ORF and thus can be viewed as a function of the RNA itself. The clones were screened for subgenomic promoter activity by inoculating the clones to MP (+) tobacco hosts. Of the 16 tested, 15 were fluorescent, indicating that the subgenomic promoter was still active in most cases. Interestingly, the apparent brightness of GFP fluorescence resulting from infection with these clones was variable from clone to clone. Examination of sequence data indicated a possible correlation between the accumulation of mutations in the subgenomic promoter region and activity of the promoter.

Screening of the chimeric movement proteins for the ability to facilitate cell-to-cell movement (Shivprasad, et al., Virology 255(1999)312-323) revealed that two of the proteins were capable of facilitating cell-to-cell spread at rates similar to wild-type TMV MP. From sequence analysis it was predicted that, upon translation of the 16 chimeric MP genes, 6 would yield nonfunctional truncated proteins because of frameshift mutations or stop codons introduced at random by PCR. Therefore only 10 of the 16 are predicted to be translated to make full-length chimeric MP proteins. It is noteworthy that the two functional clones resulted from recombination events within the same sequence region. This is not unexpected, since genes from two different species are being shuffled, and not all combinations would be expected to yield a functional product.

### EXAMPLE 3

### Molecular Breeding of the 30K Movement Proteins of Tobamoviruses II

It was found in experiments like those represented in Example 2 that the resulting shuffled clones showed a polarity in the sequence. In Example 2 the 5′ end of the shuffled clones tended to have more sequence identity to the TMV, and the 3′ end of the shuffled clones tended to have more sequence identity to the ToMV (FIG. 6). This result is likely due to the polar effect of using TMV 30K as the top strand and ToMV 30K as the bottom strand for generating the input fragments. In order to more fully sample the range of possibly improved shuffled clones, Example 2 is repeated, but using ToMV 30K to generate the top strand and TMV to generate the bottom strand. The 51′ end of the resulting shuffled clones is expected to have more sequence identity to the ToMV, and the 3′ end of the shuffled clones is expected to have more sequence identity to the TMV, similar to the results found in Example 2.

### EXAMPLE 4

### Molecular Breeding of the 30K Movement Proteins of Tobamoviruses III

The desired shuffled clones resulting from experiments like those in Example 2 and Example 3 are used to generate input strands for an additional round of shuffling. For example, in one experiment the clones from an experiment like Example 2 are used to generate top strands, and the clones from Example 3 are used to generate bottom strands. In another experiment the clones from Example 3 are used to generate top strands, and the clones from an experiment like Example 2 are used to generate bottom strands. The process of separating the desired cloned into two pools, generating top strands from one pool and bottom strands from the other is repeated as often as needed to obtain the ultimate clone.

### EXAMPLE5

### Shuffling to Generate Improved Arsenate Detoxifying Bacteria

Arsenic detoxification is important for mining of arsenopyrite-containing gold ores and other uses, such as environmental remediation. Plasmid pGJ103, containing an arsenate detoxification operon (Ji, G. and Silver, S., , J. Bacteriol. 174, 3684-3694 (1992), incorporated herein by reference), is obtained from Prof. Simon Silver (U. of Illinois, Chicago, Ill.). E. coli TG1 containing pGJ103, containing the pI258 ars operon cloned into pUC19, has a MIC (minimum inhibitory concentration) of 4 μg/ml on LB ampicillin agar plates. The ars operon is amplified by mutagenic PCR (REF), cloned into pUC19, and transformed into E. coli TG1. Transformed cells are plated on a range of sodium arsenate concentrations (2, 4, 8, 16 mM). Colonies from the plates with the highest arsenate levels are picked into one of two pools, grown in liquid in the presence of the same concentration of arsenate, and plasmid DNA is isolated separately from each pool. One pool is used to generate top strands of the ars operon by linear PCR, and the other pool is used to generate bottom strands of the ars operon by linear PCR. Shuffling and cloning of the resulting products is performed similarly to the methods used in Example 1 with primers appropriate for the ars operon and with cloning into pUC19. Resulting plasmids are transformed into E. coli TG1 and the cells are plated at higher arsenate levels; 8, 16, 32, 64 mM. Colonies are picked from the plates with the highest arsenate levels and another round of shuffling is performed as above except that resulting transformed cells are plated at 32, 64, 128, 256 mM arsenate.

Four cycles of the process are expected to result in about a 50-100-fold improvement in the resistance to arsenate conferred by the shuffled arsenate resistance operon; bacteria containing the improved operon would grow on medium containing up to about 500 mM arsenate.

### EXAMPLE 6

### Heterosexual PCR shuffling of xylE and nahH.

The xylE gene is PCR amplified from pSK-xylE using standard methods of PCR with oligonucleotide primers xylE-F (5′-GTA TAT GCG GCC GCG TGT GAG TGC ATG AAA AAA GGC GTT ATG CGA CCC GGC.-3′;SEQ ID NO:35) and xylE-R (5′-TGG ATA GAA TTC TGG CCA AAG AGA TCA GGT CAG CAC GGT CAT GAA TCG TTC.-3′;SEQ ID NO:36). The nahh gene is PCR amplified from pSK-nahH using standard methods of PCR with oligonucleotide primers nahH-F (5′-GTA TAT GCG GCC GCG TGT GAG TGC ATG AAC AAA GGT GTA ATG CGC CCC GGC.-3′; SEQ ID NO:37) and nahH-R (5′-TGG ATA GAA TTC TGG CCA AAG AGA TTA GGT CAT AAC GGT CAT GAA TCG TTC.-3′; SEQ ID NO:38). Each PCR product is gel-isolated.

To synthesize the xylE top-strand ssDNA, asymmetric PCR is performed using the xyle double-stranded PCR product as template and the primer Top-F (5′-GTA TAT GCG GCC GCG TGT GAG TGC ATG-3′; SEQ ID NO: 39) as the sole primer in the cycling reaction. The result is a mixture of double-stranded and single-stranded (top strand) DNA which is then resolved on an agarose gel. The band containing the single-stranded xylE DNA is excised and then processed using a DNA extraction spin-column.

To synthesize the nahH bottom-strand ssDNA, asymmetric PCR is performed using the nahH double-stranded PCR product as template and the primer Bottom-R (5′-TGG ATA GAA TTC TGG CCA AAG AGA TTA-3′; SEQ ID NO: 40) as the sole primer in the cycling reaction. The result is a mixture of double-stranded and single-stranded (bottom strand) DNA, which is then resolved on an agarose gel. The band containing the single-stranded nahH DNA is excised and then processed using a DNA extraction spin-column.

Separately, both ssDNAs are fragmented with an appropriate amount of DNAseI and the resulting fragments are run out on an agarose gel. The smear of DNA ranging from ˜40-100 bases is excised and processed by DNA spin-column to obtain fragments of single-stranded DNA.

The individual ssDNA samples derived from xylE and nahH are mixed and added to a reaction containing a mixture of Taq and Pfu polymerases with dNTPs and the appropriate buffer (Barnes, W. M. (1994) Proc. Natl. Acad. Sci. USA,91, 2216-2220) and cycled in a thermocycler 94° C., 30 sec, (94° C.-5 sec, 55° C.-30 sec, 72° C.-1 min)×15 cycles. An aliquot is run on a gel to confirm an increase in average size of the fragments.

One microliter of the reassembly reaction is added to a PCR mixture (similar to above) along with primers Top-F and Bottom-R, that are used to generate the ssDNAs, and cycled in a thermocycler 94° C., 30 sec. (94° C.-5 sec, 45° C.-30 sec, 72° C.-2 min)×15 cycles. An aliquot is run on a gel and the amplification product is excised and processed to isolate the DNA that is then digested with the Noti and EcoRI restriction endonucleases and inserted between the NotI and EcoRI sites of pBluescript SK (+) and introduced into E. coli TOP10F′ cells.

Greater than 14% of the resulting clones are expected to be recombinants between the xylE and nahH genes. Indeed, it is expected that essentially all of the full-length clones will be the result of recombination between these parental templates.

Throughout this application various publications have been referenced. The disclosures of these publications in their entireties are hereby incorporated by reference in this application in order to more fully describe the state of the art to which this invention pertains.

As can be appreciated from the above description, the present invention has a wide variety of applications. Variations without departing from the scope and intention of the present invention will be readily apparent to one of ordinary skill upon reviewing the above. Such variations are expected to be within the ordinary skill of the average practitioner and are encompassed by the present invention.

### Cited Reference

Arkin, A. P. and Youvan, D. C., (1992) Proc. Natl. Acad. Sci. USA 89:7811-7815.

Ausubel, et al., Current Protocols in Molecular Biology (Supplement 47), John Wiley & Sons, New York (1999).

Barnes, W. M., (1994) PNAS 91, 2216-2220.

Bartel, D. P. and Szostak, J. W., (1993) Science 261:1411-1418.

Bock, L. C., et al., (1992) Nature 355:564-566.

Cadwell, R. C. and Joyce, G. F., (1992) PCR Methods and Applications 2:28-33.

Calogero, et al., (1992) FEMS Microbiology Lett. 76: 41-44.

Caren, et al., (1994) Bio/Technology 12: 517-520.

Chang, et al., Nat Biotechnol 17(1999)793-797.

Chothia and Leks, J. Mol. Biol. 196:901-917 (1987).

Clothia, et al., Nature 342:877-833 (1989).

Coelho-Sampaio, (1993) Biochem. 32:10929-10935.

Crameri, et al., Nature 391(1998)288-291.

Cull, M. G., et al., (1992) Proc. Natl. Acad. Sci. USA 89:1865-1869.

Cwirla, S. E., et al., (1990) Proc. Natl. Acad. Sci. USA 87:6378-6382.

Delagrave, et al., (1993) Protein Engineering 6: 327-331.

Delgrave, S and Youvan, D.C., (1993) Bio/Technology 11: 1548-155.

Galizzi, et al., WO91/01087.

Goldman, E. R. and Youvan D. C., (1992) Bio/Technology 10:1557-1561.

Gram, H., et al., (1992) Proc. Natl. Acad. Sci. USA 89:3576-3580.

Hansson, et al., J Mol Biol 287(1999)265-276.

Harayama, S., Trends Biotechnol 16(1998)76-82.

Hayashi, et al., (1994) Biotechniques 17: 310-315).

Hermes, J. D. et al., (1990) Proc. Natl. Acad. Sci. USA 87:696-700.

Holland, J. H., (1992) “Adaptation in natural and artificial systems”. Second edition, MIT Press, Cambridge.

Holland, J. H., (1992) Sci. Am. July, 66-72.

Ji, G. and Silver, S., Regulation and expression of the arsenic resistance operon from Staphylococcus aureus plasmid pI258, J. Bacteriol. 174, 3684-3694 (1992).

Joyce, G. F., (1992) Scientific American 276:6 90-97.

Kabat, et al., “Sequences of Proteins of Immunological Interest”, 4th Ed., U.S. Department of Health and human services, Bethesda, MD (1987).

Kauffman, S. A., (1993) “The origins of order: self-organization and selection in evolution”. Oxford University Press, New York.

Kikuchi, et al., Gene 236:159-167 (1999)

Kikuchi, et al., Gene 243:133-137 (2000).

Kimmel and Berger, Methods Enzymol. Vol. 152. Guide to Molecular Cloning Techniques (1987), Academic Press, Inc., San Diego, Calif.

Kumamaru, et al., Nat Biotechnol 16(1998)663-666.

Marton, et al., Nucleic Acids Res. 19(1991)2423-2426.

Matsushita, et al., J Gen Virol 81(2000)2095-2102.

McCabe, Peter C., (1990) Production of single stranded DNA by asymmetric PCR in PCR Protocols: A guide to methods and applications. eds. M. A. Innis, D. H. Gelfand, J. J. Sninsky and T. J. White. Academic Press Inc., San Diego, Calif. USA.

McCafferty, J., et al., (1990) Nature 348:552-554.

Meyerhans, et al., (1990) Nucleic Acids Res. 18:1687-1691.

Needleman and Wunsch, J. Mol. Biol. 48:443 (1970).

Nissim, et al., (1994) EMBO J. 13: 692-698.

Oliphant, A. R., et al., (1986) Gene 44:177-183.

Oliver, et al., Virol 155(1986)277-283.

Patten, et al., Curr Opin Biotechnol 8(1997)724-733.

Pearson and Lipman, Proc. Natl. Acad. Sci. U.S.A. 85:2444-2448 (1988).

Radman, et al., WO90/07576.

Reidhaar-Olson, J. F and Sauer, R. T., (1988) Science 241:53-57.

Sambrook, et al., Molecular Cloning: A Laboratory Manual, 2nd ed., Cold Spring Harbor Press, Plainview, N.Y. (1989).

Schneider, T. D., et al., (1986) J. Mol. Biol. 188:415-431.

Scott, J. K. and Smith, G. P., (1990) Science 249:386-390.

Shivprasad, et al., Virol 255(1999)312-323.

Smith and Waterman, Adv. Appl. Math. 2:482 (1981).

Stemmer, W. P.: DNA shuffling by random fragmentation and reassembly: in vitro recombination for molecular evolution. Proc. Natl. Acad. Sci. USA (1994) 91:10747-10751.

Stemmer W. P.: Rapid evolution of a protein in vitro by DNA shuffling: Nature 370 (1994) 389-391

Stemmer, W. P. C., et al., (1993) Biotechniques 14:256-265.

Stormo, G. D., (1991) Methods Enzymol. 208:458-468.

Tramontano, et al., J. Mol. Biol. 215:175-182 (1990).

U.S. Pat. No. 5,605,793

U.S. Pat. No. 5,811,238

U.S. Pat. No. 5,830,721

U.S. Pat. No. 5,928,905

U.S. Pat. No. 6,096,548

U.S. Pat. No. 6,117,679

U.S. Pat. No. 6,165,793

U.S. Pat. No. 6,153,410.

U.S. Pat. No. 4,683,202.

U.S. Pat. No. 4,683,195.

U.S. Pat. No. 6,096,548.

Weber, et al., J Virol 66(1992)3909-3912.

Williams, A. F. and A. N. Barclay, “The Immunoglobulin Gene Superfamily”, in Immunoglobulin Genes, T. Honjo, F. W. Alt, and T. H. Rabbitts eds., (1989) Academic Press: San Diego, Calif., pp. 361-368.

Winter, et al., (1994) Ann. Rev. Immunol. 12: 433-55.

Yockey, H. P., (1977) J. Theor. Biol. 67:345-376.

Yockey, H. P., (1974) J. Theor. Biol. 46:369-380.

What is claimed is:

1. A method of forcing recombination between polynucleotides, comprising:
(a) generating a single strand of a first polynucleotide;
(b) generating a single strand of a second polynucleotide, wherein said second polynucleotide is partially complementary to said first polynucleotide;
(c) fragmenting said single strand of said first polynucleotide to generate single stranded first polynucleotide fragments, and optionally isolating a size range of single stranded fragments;
(d) fragmenting said single strand of said second polynucleotide to generate single stranded second polynucleotide fragments, and optionally isolating a size range of single stranded fragments;
(e) annealing said single stranded first polynucleotide fragments with said single stranded second polynucleotide fragments; and
(f) extending said annealed polynucleotide fragments.

2. The method of claim 1, further comprising adding at least one additional single stranded polynucleotide partially complementary to said first or second polynucleotide.

3. The method of claim 1, further comprising
(g) denaturing said extended annealed polynucleotide fragments; and
(h) repeating steps (e) and (f).

4. The method of claim 3, wherein steps (g) and (h) are repeated one or more times, wherein a larger polynucleotide is generated.

5. The method of any of claims 1 or 3, further comprising the steps of:
(i) generating a single strand of a third polynucleotide, wherein said third polynucleotide is the exact complement of said first polynucleotide;
(j) generating a single strand of a fourth polynucleotide, wherein said fourth polynucleotide is the exact complement of said second polynucleotide;
(k) fragmenting said single strand of said third polynucleotide to generate single stranded third polynucleotide fragments, and optionally isolating a size range of single stranded fragments;
(l) fragmenting said single strand of said fourth polynucleotide to generate single stranded fourth polynucleotide fragments, and optionally isolating a size range of single stranded fragments;
(m) annealing said single stranded third polynucleotide fragments with said single stranded fourth polynucleotide fragments; and
(n) extending said annealed polynucleotide fragments.

6. The method of any of claims 1, 3 or 4, further comprising isolating a size range of double stranded polynucleotides.

7. The method of claim 6, further comprising adding primers and amplifying all or a portion of said extended annealed fragments.

8. The method of any of claims 1 through 4, further comprising adding primers and amplifying all or a portion of said extended annealed fragments.

9. The method of claim 3, further comprising selecting a recombinant polynucleotide to identify a recombinant nucleotide having a desired functional property.

10. The method of claim 3, further comprising screening a recombinant polynucleotide for a desired functional property.

11. The method of claim 1, wherein fragmenting is performed enzymatically, chemically or physically.

12. The method of claim 1, wherein said single strand of said first polynucleotide and said second polynucleotide is generated by asymmetric PCR or single stranded nucleic acid vector.

13. The method of any of claims 1 or 2, further comprising adding a first clamp to the 5′ end of said first single stranded polynucleotide, and any other polynucleotides of the same sense, and a second clamp to the 5′ end of said second polynucleotide, and any other polynucleotides of the complementary sense of said first single stranded polynucleotide.

14. A method of forcing recombination between polynucleotides, comprising:
(a) fragmenting a single stranded first polynucleotide and a single stranded second polynucleotide to generate single stranded first and second polynucleotide fragments, wherein said second polynucleotide is partially complementary to said first polynucleotide, and optionally isolating a size range of single stranded fragments;
(b) annealing said single stranded first polynucleotide fragments with said single stranded second polynucleotide fragments; and
(c) extending said annealed fragments.

15. The method of claim 14, further comprising adding at least one additional single stranded polynucleotide partially complementary to said first or second polynucleotide.

16. The method of claim 14, further comprising
(d) denaturing said extended annealed fragments; and
(e) repeating steps (b) and (c) one or more times, thereby generating one or more recombinant polynucleotides.

17. The method of claim 16, wherein steps (d) and
(e) are repeated one or more times, wherein a full-length polynucleotide is generated.

18. The method of any of claims 14 or 16, further comprising the steps of:
(f) fragmenting a single stranded third polynucleotide, wherein said third polynucleotide is the exact complement of said first polynucleotide, and a single stranded fourth polynucleotide, wherein said fourth polynucleotide is the exact complement of said second polynucleotide, to generate single stranded third and fourth polynucleotide fragments, and optionally isolating a size range of single stranded fragments;
(g) annealing said single stranded third polynucleotide fragments with said single stranded fourth polynucleotide fragments; and
(h) extending said annealed fragments.

19. The method of any of claims 14, 16, or 17, further comprising isolating a size range of double stranded polynucleotide.

20. The method of claim 19, further comprising adding primers and amplifying all or a portion of said extended annealed fragments.

21. The method of any of claims 14 through 17, further comprising adding primers and amplifying all or a portion of said extended annealed fragments.

22. The method of claim 16, further comprising selecting a recombinant polynucleotide to identify a recombinant nucleotide having a desired functional property.

23. The method of claim 16, further comprising screening a recombinant polynucleotide for a desired functional property.

24. The method of claim 14, wherein fragmenting is performed enzymatically, chemically or physically.

25. The method of claim 14, wherein said single strand of said first polynucleotide and said second polynucleotide are generated by asymmetric PCR or single stranded nucleic acid vector.

26. The method of claim 14, further comprising adding a first clamp to the 5′ end of said first single stranded polynucleotide, and any other polynucleotides of the same sense, and a second clamp to the 5′ end of said second polynucleotide, and any other polynucleotides of the complementary sense of said first single stranded polynucleotide.

27. A method of forcing recombination between polynucleotides, comprising:
(a) generating a single strand of a first polynucleotide;
(b) generating a single strand of a second polynucleotide, wherein said second polynucleotide is partially complementary to said first polynucleotide;
(c) annealing said single strands of said first and second polynucleotides;
(d) fragmenting said annealed polynucleotides to generate partially double stranded polynucleotide fragments, and optionally isolating a size range of single stranded fragments;
(e) denaturing said partially double stranded polynucleotide fragments;
(f) annealing said denatured polynucleotide fragments; and
(g) extending said annealed polynucleotide fragments.

28. The method of claim 27, further comprising adding at least one additional single stranded polynucleotide partially complementary to said first or second polynucleotide.

29. The method of claim 27, further comprising
(h) denaturing said extended fragments;
(i) annealing said denatured extended fragments; and
(j) extending said annealed extended fragments.

30. The method of claim 29, wherein steps (h) through (j) are repeated one or more times, wherein a full-length polynucleotide is generated.

31. The method of any of claims 27 or 29, further comprising the steps of:
(k) generating a single strand of a third polynucleotide, wherein said third polynucleotide is the exact complement of said first polynucleotide;
(l) generating a single strand of a fourth polynucleotide, wherein said fourth polynucleotide is the exact complement of said second polynucleotide;
(m) annealing said single strands of said third and fourth polynucleotides;
(n) fragmenting said annealed polynucleotides to generate partially double stranded polynucleotide fragments, and optionally isolating a size range of single stranded fragments;
(o) denaturing said partially double stranded polynucleotide fragments;
(p) annealing said denatured polynucleotide fragments; and
(q) extending said annealed polynucleotide fragments.

32. The method of any of claims 25, 27 or 28, further comprising isolating a size range of double stranded polynucleotide.

33. The method of claim 32, further comprising adding primers and amplifying all or a portion of said extended fragments.

34. The method of any of claims 27 through 30, further comprising adding primers and amplifying all or a portion of said extended fragments.

35. The method of claim 30, further comprising selecting a recombinant polynucleotide to identify a recombinant nucleotide having a desired functional property.

36. The method of claim 29, further comprising screening a recombinant polynucleotide for a desired functional property.

37. The method of claim 27, wherein fragmenting is performed enzymatically, chemically or physically.

38. The method of claim 27, wherein said single strand of said first polynucleotide and said second polynucleotide is generated by asymmetric PCR or single stranded nucleic acid vector.

39. The method of claim 27 or 28, further comprising adding a first clamp to the 5′ end of said first single stranded polynucleotide, and any other polynucleotides of the same sense, and a second clamp to the 5′ end of said second polynucleotide, and any other polynucleotides of the complementary sense of said first single stranded polynucleotide.

40. A method of forcing recombination between polynucleotides, comprising:
(a) annealing a single stranded first polynucleotide with a single stranded second polynucleotide, wherein said second polynucleotide is partially complementary to said first polynucleotide,
(b) fragmenting said annealed polynucleotides to generate partially double stranded polynucleotide fragments;
(c) denaturing said partially double stranded polynucleotide fragments;
(d) annealing said denatured polynucleotide fragments; and
(e) extending said annealed polynucleotide fragments.

41. The method of claim 40, further comprising adding at least one additional single stranded polynucleotide partially complementary to said first or second polynucleotide.

42. The method of claim 40, further comprising
(f) denaturing said extended polynucleotide;
(g) annealing said denatured extended polynucleotide; and
(h) extending said annealed extended fragments.

43. The method of claim 41, wherein steps (g) through (h) are repeated one or more times, wherein a full-length polynucleotide is generated.

44. The method of any of claims 40 or 42, further comprising the steps of:
(i) annealing a single stranded third polynucleotide with a single stranded fourth polynucleotide, wherein said third polynucleotide is the exact complement of said first polynucleotide and said fourth polynucleotide is the exact complement of said second polynucleotide;
(j) fragmenting said annealed polynucleotides to generate partially double stranded polynucleotide fragments;
(k) denaturing said partially double stranded polynucleotide fragments;
(l) annealing said denatured polynucleotide fragments; and
(m) extending said annealed polynucleotide fragments.

45. The method of claims 40, or 42 through 43, further comprising isolating a size range of double stranded polynucleotide.

46. The method of claim 45, further comprising adding primers and amplifying all or a portion of said extended fragments.

47. The method of any of claims 40 through 43, further comprising adding primers and amplifying all or a portion of said extended fragments.

48. The method of claim 47, further comprising selecting a recombinant polynucleotide having a desired functional property.

49. The method of claim 47, further comprising screening a recombinant polynucleotide for a desired functional property.

50. The method of claim 40, wherein fragmenting is performed enzymatically, chemically or physically.

51. The method of claim 40, wherein said single strand of said first polynucleotide and said second polynucleotide is generated by asymmetric PCR or single stranded nucleic acid vector.

52. The method of claim 40 or 41, further comprising adding a first clamp to the 5′ end of said first single stranded polynucleotide, and any other polynucleotides of the same sense, and a second clamp to the 5′ end of said second polynucleotide, and any other polynucleotides of the complementary sense of said first single stranded polynucleotide.

53. A method of forcing recombination between polynucleotides, comprising:
(a) generating a single strand of a first polynucleotide;
(b) generating a single strand of a second polynucleotide, wherein said second polynucleotide is partially complementary to said first polynucleotide;
(c) generating a single strand of a third polynucleotide;
(d) fragmenting said single strand of said first polynucleotide to generate single stranded first polynucleotide fragments, and optionally isolating a size range of single stranded fragments;
(e) fragmenting said single strand of said second polynucleotide to generate single stranded second polynucleotide fragments, and optionally isolating a size range of single stranded fragments;
(f) fragmenting said single strand of said third polynucleotide to generate single stranded third polynucleotide fragments, and optionally isolating a size range of single stranded fragments;
(g) annealing said single stranded first polynucleotide fragments with said single stranded second polynucleotide fragments;
(h) extending said annealed polynucleotide
(i) denaturing said extended annealed polynucleotide fragments;
(j) annealing said single stranded third polynucleotide fragments with said extended, annealed, and denatured polynucleotide fragments from step (g); and
(k) extending said annealed polynucleotide fragments.

54. The method of claim 53 wherein said single stranded third polynucleotide has greater sequence homology to said single stranded first polynucleotide than to said single stranded second polynucleotide.

55. The method of claim 53 wherein said single stranded third polynucleotide has greater sequence homology to said single stranded second polynucleotide than to said single stranded first polynucleotide.

56. The method of claim 53, further comprising adding at least one additional single stranded polynucleotide partially complementary to said first, second, or third polynucleotide.

57. The method of claim 53, further comprising:
(l) denaturing said extended annealed polynucleotide fragments; and
(m) repeating steps (j), and(k).

58. The method of claim 57, wherein steps (1) and (m) are repeated one or more times, wherein a larger polynucleotide is generated.

59. The method of claim 53, further comprising:
(n) generating a single strand of a fourth polynucleotide, wherein said fourth polynucleotide is the exact complement of said first polynucleotide;
(o) generating a single strand of a fifth polynucleotide, wherein said fifth polynucleotide is the exact complement of said second polynucleotide;
(p) generating a single strand of a sixth polynucleotide, wherein said sixth polynucleotide is the exact complement of said third polynucleotide;
(q) fragmenting said single strand of said fourth polynucleotide to generate single stranded fourth polynucleotide fragments, and optionally isolating a size range of single stranded fragments;
(r) fragmenting said single strand of said fifth polynucleotide to generate single stranded fifth polynucleotide fragments, and optionally isolating a size range of single stranded fragments;
(s) fragmenting said single strand of said sixth polynucleotide to generate single stranded sixth polynucleotide fragments, and optionally isolating a size range of single stranded fragments;
(t) annealing said single stranded fourth polynucleotide fragments with said single stranded fifth polynucleotide fragments;
(u) extending said annealed polynucleotide fragments;
(v) denaturing said extended annealed polynucleotide fragments;
(w) annealing said single stranded sixth polynucleotide fragments with said extended, annealed, and denatured polynucleotide fragments from step (t); and
(x)extending said annealed polynucleotide fragments.

60. The method of any of claims 53, 57 or 58, further comprising isolating a size range of double stranded polynucleotides after either step (f), step (k), step (l) or step (m).

61. The method of claim 60, further comprising adding primers and amplifying all or a portion of said extended annealed fragments.

62. The method of any of claims 53 through 58, further comprising adding primers and amplifying all or a portion of said extended annealed fragments.

63. The method of claim 57, further comprising selecting a recombinant polynucleotide to identify a recombinant nucleotide having a desired functional property.

64. The method of claim 57, further comprising screening a recombinant polynucleotide for a desired functional property.

65. The method of claim 53, wherein fragmenting is performed enzymatically, chemically or physically.

66. The method of claim 53, wherein said single strand of said first polynucleotide and said second polynucleotide is generated by asymmetric PCR or single stranded nucleic acid vector.

67. The method of claim 53 or 56, further comprising adding a first clamp to the 5′ end of said first single stranded polynucleotide, and any other polynucleotides of the same sense, and a second clamp to the 5′ end of said second polynucleotide, and any other polynucleotides of the complementary sense of said first single stranded polynucleotide.

68. A method of forcing recombination between polynucleotides, comprising:
(a) generating a single strand of a first polynucleotide;
(b) generating a single strand of a second polynucleotide, which is partially complementary to said first polynucleotide;
(c) generating a fragmented single strand of a third polynucleotide, which is partially complementary to said first polynucleotide or second polynucleotide;
(d) annealing said single strands of said first and second polynucleotides;
(e) fragmenting said annealed polynucleotides to generate partially double stranded polynucleotide fragments;
(f) denaturing said partially double stranded polynucleotide fragments;
(g) annealing said denatured polynucleotide fragments;
(h) extending said annealed polynucleotide fragments;
(i) denaturing said partially double stranded polynucleotide fragments;
(j) adding said fragmented single strand of said third polynucleotide to denatured partially double stranded polynucleotide fragments;
(k) annealing said fragmented single strand of said third polynucleotide either to extended first polynucleotide fragments or to extended second polynucleotide fragments;
(l) denaturing said partially double stranded polynucleotide fragments;
(m) annealing said denatured polynucleotide fragments; and
(n) extending said annealed polynucleotide fragments.

69. The method of claim 68, further comprising adding at least one additional single stranded polynucleotide partially complementary to said first or second polynucleotide.

70. The method of claim 68, further comprising:
(o)denaturing said extended fragments;
(p)annealing said denatured extended fragments; and
(q)extending said annealed extended fragments.

71. The method of claim 73, wherein steps (o) through (q) are repeated one or more times, wherein a full-length polynucleotide is generated.

72. The method of any of claims 70 or 71, further comprising:
(r) generating a single strand of a fourth polynucleotide, wherein said fourth polynucleotide is the exact complement of said first polynucleotide;
(s) generating a single strand of a fifth polynucleotide, wherein said fifth polynucleotide is the exact complement of said second polynucleotide;
(t) generating a fragmented single strand of a sixth polynucleotide, wherein said sixth polynucleotide is the exact complement of said third polynucleotide;
(u) annealing said single strands of said fourth and fifth polynucleotides;
(v) fragmenting said annealed polynucleotides to generate partially double stranded polynucleotide fragments;
(w) denaturing said partially double stranded polynucleotide fragments;
(x) annealing said denatured polynucleotide fragments;
(y) extending said annealed polynucleotide fragments;
(z) denaturing said partially double stranded polynucleotide fragments;
(aa) adding said fragmented single strand of said sixth polynucleotide to denatured partially double stranded polynucleotide fragments;
(bb) annealing said fragmented single strand of said sixth polynucleotide either to extended fourth polynucleotide fragments or to extended fifth polynucleotide fragments;
(cc) denaturing said partially double stranded polynucleotide fragments;
(dd) annealing said denatured polynucleotide fragments; and
(ee) extending said annealed polynucleotide fragments.

73. The method of any of claims 68, 70 or 71, further comprising isolating a size range of double stranded polynucleotide after step (e), step (h), step (m), or step (q).

74. The method of claim 73, further comprising adding primers and amplifying all or a portion of said extended fragments.

75. The method of any of claims 68 through 71, further comprising adding primers and amplifying all or a portion of said extended fragments.

76. The method of claim 68 or 70, further comprising selecting a recombinant polynucleotide to identify a recombinant nucleotide having a desired functional property.

77. The method of claim 68 or 70, further comprising screening a recombinant polynucleotide for a desired functional property.

78. The method of claim 68, wherein fragmenting is performed enzymatically, chemically or physically.

79. The method of claim 68, wherein said single strand of said first polynucleotide and said second polynucleotide is generated by asymmetric PCR or single stranded nucleic acid vector.

80. The method of any of claims 68 or 71, further comprising adding a first clamp to the 5′ end of said first single stranded polynucleotide, and any other polynucleotides of the same sense, and a second clamp to the 5′ end of said second polynucleotide, and any other polynucleotides of the complementary sense of said first single stranded polynucleotide.

81. The method of claim 68 further comprising extending said partially double stranded polynucleotide fragments after step (e).

82. A method of forcing recombination between polynucleotides, comprising:
(a) generating a single strand of a first polynucleotide;
(b) generating a single strand of a second polynucleotide, which is partially complementary to said first polynucleotide;
(c) generating a single strand of a third polynucleotide, which is partially complementary to said first polynucleotide or second polynucleotide;
(d) annealing said single strands of said first and second polynucleotides;
(e) fragmenting said annealed polynucleotides to generate partially double stranded polynucleotide fragments;
(f) denaturing said partially double stranded polynucleotide fragments;
(g) annealing said denatured polynucleotide fragments;
(h) extending said annealed polynucleotide fragments;
(i) denaturing said partially double stranded polynucleotide fragments;
(j) adding said single strand of said third polynucleotide to denatured partially double stranded polynucleotide fragments;
(k) annealing said single strand of said third polynucleotide either to extended first polynucleotide fragments or to extended second polynucleotide fragments;
(l) fragmenting said annealed polynucleotides to generate partially double stranded polynucleotide fragments, and optionally isolating a size range of single stranded fragments;
(m) denaturing said partially double stranded polynucleotide fragments;
(n) annealing said denatured polynucleotide fragments; and
(o) extending said annealed polynucleotide fragments.

83. The method of claim 82, further comprising adding at least one additional single stranded polynucleotide partially complementary to said first or second polynucleotide.

84. The method of claim 82, further comprising:
(p)denaturing said extended fragments;
(q)annealing said denatured extended fragments; and
(r)extending said annealed extended fragments.

85. The method of claim 83, wherein steps (p) through (r) are repeated one or more times, wherein a full-length polynucleotide is generated.

86. The method of any of claims 82 or 84, further comprising
(s) generating a single strand of a fourth polynucleotide, wherein said fourth polynucleotide is the exact complement of said first polynucleotide;
(t) generating a single strand of a fifth polynucleotide, wherein said fifth polynucleotide is the exact complement of said second polynucleotide;
(u) generating a single strand of a sixth polynucleotide, wherein said sixth polynucleotide is the exact complement of said third polynucleotide;
(v) annealing said single strands of said fourth and fifth polynucleotides;
(w) fragmenting said annealed polynucleotides to generate partially double stranded polynucleotide fragments;
(x) denaturing said partially double stranded polynucleotide fragments;
(y) annealing said denatured polynucleotide fragments;
(z) extending said annealed polynucleotide fragments;
(aa) denaturing said partially double stranded polynucleotide fragments;
(bb) adding said single strand of said sixth polynucleotide to denatured partially double stranded polynucleotide fragments;
(cc) annealing said single strand of said sixth polynucleotide either to extended fourth polynucleotide fragments or to extended fifth polynucleotide fragments;
(dd) fragmenting said annealed polynucleotides to generate partially double stranded polynucleotide fragments, and optionally isolating a size range of single stranded fragments;
(ee) denaturing said partially double stranded polynucleotide fragments;
(ff) annealing said denatured polynucleotide fragments; and
(gg) extending said annealed polynucleotide fragments.

87. The method of any of claims 82, 84 or 85, further comprising isolating a size range of double stranded polynucleotide after step (e), step (h), step (m) or (r).

88. The method of claim 87, further comprising adding primers and amplifying all or a portion of said extended fragments.

89. The method of any of claims 82 through 85, further comprising adding primers and amplifying all or a portion of said extended fragments.

90. The method of claim 82 or 84, further comprising selecting a recombinant polynucleotide to identify a recombinant nucleotide having a desired functional property.

91. The method of claim 82 or 84, further comprising screening a recombinant polynucleotide for a desired functional property.

92. The method of claim 82, wherein fragmenting is performed enzymatically, chemically or physically.

93. The method of claim 82, wherein said single strand of said first polynucleotide and said second polynucleotide is generated by asymmetric PCR or single stranded nucleic acid vector.

94. The method of claim 82 or 83, further comprising adding a first clamp to the 5′ end of said first single stranded polynucleotide, and any other polynucleotides of the same sense, and a second clamp to the 5′ end of said second polynucleotide, and any other polynucleotides of the complementary sense of said first single stranded polynucleotide.

95. The method of claim 82 further comprising extending said partially double stranded polynucleotide fragments after step (e).


SEQUENCE LISTING (40 sequences)

SEQ ID NO: 1 - DNA, 10 bp, Artificial Sequence
  exemplary only
agatcaattg

SEQ ID NO: 2 - DNA, 10 bp, Artificial Sequence
  exemplary only
caattgatct

SEQ ID NO: 3 - DNA, 10 bp, Artificial Sequence
  exemplary only
agaccgattg

SEQ ID NO: 4 - DNA, 10 bp, Artificial Sequence
  exemplary only
caatcggtct

SEQ ID NO: 5 - DNA, 21 bp, Artificial Sequence
  primer
gaagaagtcgttgatgagttc

SEQ ID NO: 6 - DNA, 23 bp, Artificial Sequence
  primer
gctttattacgtgcctgcggatg

SEQ ID NO: 7 - DNA, 25 bp, Artificial Sequence
  primer
atgttaaggattttggaggaatgag

SEQ ID NO: 8 - DNA, 36 bp, Artificial Sequence
  primer
cgttatttcctaggcatcttgactacccctcgagtg

SEQ ID NO: 9 - DNA, 58 bp, Artificial Sequence
  primer
ggcacaagttacaagcagtcacgcaaccctaggatggctctagttgttaaaggaaaag

SEQ ID NO: 10 - DNA, 57 bp, Artificial Sequence
  primer
cagcggcttagcctaagcaaaattaattgtcgtcattcacagataaatgtcccatca

SEQ ID NO: 11 - DNA, 58 bp, Artificial Sequence
  primer
ggcacaagttacaagcagtcacgcaaccctaggatggctctagttgttaaaggtaagg

SEQ ID NO: 12 - DNA, 57 bp, Artificial Sequence
  primer
cagcgcctaagactaagcataattaattgacgtcattcacagataaatgtcccatca

SEQ ID NO: 13 - DNA, 58 bp, Artificial Sequence
  primer
gtacgtagtcacgacattagagtaacacgcgtgaggcacaagttacaagcagtcacgc

SEQ ID NO: 14 - DNA, 58 bp, Artificial Sequence
  primer
cattcacagataaatgtcccatcagtacatatttaggcatagttactcacaagataag

SEQ ID NO: 15 - DNA, 22 bp, Artificial Sequence
  primer
gtacgtagtcacgacattagag

SEQ ID NO: 16 - DNA, 23 bp, Artificial Sequence
  primer
ggcatagttactcacaagataag

SEQ ID NO: 17 - DNA, 807 bp, tobamovirus
atggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcagaggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtaatgatcggtcagtgccgaacaagaactatagaaatgttaaggattttggaggaatgagttttaaaaagaataatttaatcgatgatgattcggaggctactgtcgccgaatcggattcgttttaa

SEQ ID NO: 18 - DNA, 795 bp, tobamovirus
atggctctagttgttaaaggtaaggtaaatattaatgagtttatcgatctgtcaaagtctgagaaacttctcccgtcgatgttcacgcctgtaaagagtgttatggtttcaaaggttgataagattatggtccatgaaaatgaatcattgtctgaagtaaatctcttaaaaggtgtaaaacttatagaaggtgggtatgtttgcttagtcggtcttgttgtgtccggtgagtggaatttaccagataattgccgtggtggtgtgagtgtctgcatggttgacaagagaatggaaagagcggacgaagccacactggggtcatattacactgctgctgctaaaaagcggtttcagtttaaagtggtcccaaattacggtattacaacaaaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcggaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaa

SEQ ID NO: 19 - DNA, 805 bp, tobamovirus
cctaggatgcctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgagaaacgtctggcaagttttagttaatattagaaatgtgaagatgtcagcgggtttctgtccgctttctctggagtttgtgtcggtgtgtattgtttataaaaataatataaaattgggtttgagggagaaagtagcgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcagattctgattcgtattaattaa

SEQ ID NO: 20 - DNA, 805 bp, tobamovirus
cctaggatggctctagttgttaaaggaaaagtgaatatcaatgagtttatcgacctgacaaaaatggagaagatcttaccgtcgatgtttacccctgtaaagagtgttatgtgttccgaagttgataaaataatggtccatgaaaatgaatcattgtctgaagtaaatctcttaaaaggtgtaaaacttatagaaggtgggtatgtttgcttagttggtcttgttgtgtccggtgagtggaatttaccagataattgccgtggtggtgtgagtgtctgcatggttgacaagagaatggaaagagcggacgaagccacactggtgtcatattacactgctgctgctaaaaagcggtttcagtttaaagtggtcccaaattacggtattactacaaaggatgcagaaaagaacataaggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 21 - DNA, 805 bp, tobamovirus
cctaggatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagtgtctgcatggttgacaagagaatggaaagagcggacgaagccacactggggtcatattacactgctgctgctaaaaagcggtttcagtttaaagtggtcccaaattacggtattactacaaaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgctccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 22 - DNA, 806 bp, tobamovirus
cctaggatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagtgtctgcatggttgacaagagaatggaaagagcggacgaagccacactggggtcatattacactgctgctgctaaaaagcggtttcagtttaaagtggtcccaaattacggtattactacaaaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggaccaatggaactttcagaagaagttgttgatgagttcatggagaatgttccaatatcggttagactcgcaaagtttcgaaccaaatcctcaaaaagaggtacgaaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 23 - DNA, 805 bp, tobamovirus
cctaggatggctctagttgttaaaggaaaagtgaatatcaatgagtttatcgacctgacaaaaatagagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacactgctgctgctaaaaagcggtttcagtttaaagtggtcccaaattacggtattactacaaaggatgcagaaaagaacatatggcaggtcttagtaaatattaaaaatgtaaaaatgagtgcgggctactgccctttgtcattagaatttgtgtctgtgtgtattgtttataaaaatgatataaaattgggtttgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 24 - DNA, 802 bp, tobamovirus
cctaggatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagagtgagtcattgtcaggggtgaaccttcttaaaggagttaagcttattgatagtggatacgtccgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgagaaacgtctggcaagttttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacatggaactttcagaagaagttgttgatgagttcatggagaatgttccaatgtcggttagactcgcaaagtttcgaaccaaatcctcaaaaagaggtccgaaaaataataataatttggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 25 - DNA, 805 bp, tobamovirus
cctaggatggctctagttgttaaaggaaaagtgaatatcaatgagtttatcgacctgacaaaaatggagaagatcctaccgtcgatgtttacccctgtaaagagtgttatgtgttccaaagttgataaaataatggttcatgagaatgagtcattgtcaggggtgaaccttcttaaaggagttaagcttattgatagtggatacgtctgttttgccggtttggtcgtcacgggcgagtggaacttgcctgacaattgcagaggaggtgtgagcgtgtgtctggtggacaaaaggatagaaagagccgacgaggccattctcggatcttactacacagcagctgcaaagaaaagatttcagttcaaggtcgttcccaattatgctataaccacccaggacgcgatgagaaacgtctggcaagttttagctaatattagaaatgtgaagatgtcagcgggtttctgtccgctttctctggagtttgtgtcggtgtgtattgtttataaaaataatataaaattgggtctgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgccaaagagtttgataatttgattgaagatgaagccgagacgtcggttgcggattctgattcgtattaattaa

SEQ ID NO: 26 - DNA, 805 bp, tobamovirus
cctaggatggctctcgttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaatttaccagataattgccgtggtggtgtgagtgtctgcatggttgacaagagaatggaaagagcggacgaagccacactggggtcatattacactgctgctgctaaaaagcggtttcagtttaaagtggtcccaaattacggtactactacaaaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcggttaggctcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 27 - DNA, 805 bp, tobamovirus
cctaggatgtctctagttgttaaaggaaaagtgaatatcaatgagtttatcgacctgacaaaaatggagaagatcttaccgtcgatgtttacccctgtaaggagtgttatgtgttccaaagttgataaaacaatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgagaaacgtctggcaagttttagttaatattagaaatgtgaagatgtcagcggatttctgtccgctttctctggagtttgtgtcggtgtgtattgtttatagaaataatataaaattaggtttgagagagaagattacaaacgtgagagacggagggcccatggaactttcagaagaagttgttgatgagttcatggagaatgttccaaagtcggttagactcgcaaagtttcgaaccaaatcctcaaaaagaggtccgaaaaataataataatttaggtaaggggcgttcaggcggaaggcctaaaccaaaaagttttgatggagttgaaaaagagtttgataatttgattgaagatgaagccgagacgtcggtcgcggattctgattcgtattaattaa

SEQ ID NO: 28 - DNA, 804 bp, tobamovirus
cctaggatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgccagataattgccgtggtggtgtgagtgtctgcatggttgacaagagaatggaaagagcggacgaagccacactggggtcatattacactgctgctgctaaaaagcggtttcagtttaaagtggtcccaaattacggtattactacaaaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggaccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatggagccgagacgtcggtcgcggattctgattcgtattaattaa

SEQ ID NO: 29 - DNA, 804 bp, tobamovirus
cctaggatgcctctagttgttaaaggaaaagtgaatatcaatgagtttaccgacctgacaaaaatggagaagatcttaccgtcgatgtttacccctgtaaagagtgttatgtgttccaaagttgataaaataatggttcatgaaaatgaatcattgtctgaagtaaatctcttaaaaggtgtaaaacttatagaaggtgggtatgtttgcttagttggtcttgttgtgtccggtgagtggaatttacagataattgcagaggaggtgtgagcgtgtgtctggtggacaaaaggatggaaagagcggacgaagccacactggggtcatattgcactgctgctgctaaaaagcggtttcagtttaaagtggtcccaaattacggtattactacaaaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgaggcgtcggtcgcggattctgattcgtattaattaa

SEQ ID NO: 30 - DNA, 804 bp, tobamovirus
cctaggatgcctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttaatacacagcagctgcaaagaaaagatttcagttcaaggtcgttcccaattatgctataaccacccaggacgcgaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcggttagactcgcaaagtttcgagccaaatcctcaaaaagaggtccgaaaaacaataataatttaggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 31 - DNA, 805 bp, tobamovirus
cctaggatggctctaattgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaagagaatggaaagagcggacgaagccacactggggtcatattacactgctgctgctaaaaagcggtttcagtttaaagtggtcccaaattacggtattactacaaaggatgcagaaaagaacatatggcaggtcttagtaaatattaaaaatgtaaaaatgagtgcgggctactgccctttgtcattagaatttgtgtctgtgtgtattgtttataaaaataatataaaattgggtttgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 32 - DNA, 804 bp, tobamovirus
cctaggatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccattctcggatcttactacacagcagctgcaaagaaaagatttcagttcaaggtcgttcccaattatgctataaccacccaggacgcgatgagaaacgtctggcaagttttagttaatattagaaatgtgaagatgtcagcgggtttctgtccgctttctctggagtttgtgtcggtgtgtattgtttataaaaataatataaaattgggtttgagggagaaagtaacgagtgtgaacgatggaggacccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcgattctgattcgtattaattaa

SEQ ID NO: 33 - DNA, 805 bp, tobamovirus
cctaggatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtgggtacgtctgtttagccggtttggtcgtcacgggcgagtggaacttgcctgacaattgccgtggtggtgtgagtgtctgcatggttgacaagagaatggaaagagcggacgaagccacactggggtcatattacactgctgctgctaaaaagcggtttcagtttaaagtggtcccaaattacggtattactacaaaggatgcagaaaagagcatatggcaggtcttagtaaatattaaaaatgtaaaaatgagtgcgggctactgccctttgtcattaggatttgtgtctgtgtgtattgtttataaaaataatataaaattgggtttgagggagaaagtaacgagtgtgaacgatggaggacccatggaactttcagaagaagttgttgatgagttcatggagaatgttccaatatcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcggattctgattcgtattaattaa

SEQ ID NO: 34 - DNA, 804 bp, tobamovirus
cctaggatggctctagttgtcaaaggaaaagtgaatatcaatgagtctatcgacctgacaaaaatggagaagatcttaccgtcgatgtttacccctgtaaagagtgttatgtgttccaaagttgataaaataatggttcatgagaatgagtcattgtcaggggtgaaccttcttaaaggagttaagcttattgatagtggatacgtctgtttagccggtttggtcgtcacgggcgagtggaacttgcctgacaattgcagaggaggtgtgagcgtgtgtctggtggacaaaggatggaaagagccgacgaggccattctcggatcttactacacagcagctgcaaagaaaagatttcagttcaaggtcgttcccaattatgctataaccacccaggacgcgatgagaaacgtctggcaagttttagttaatattagaaatgtgaagatgtcagcgggtttctgtccgctttctctggagtttgtgtcggtgtgtattgtttataaaaataatataaaattgggtttgagggagaaagtaacgagtgtgaacgatggagggcccatggaactttcagaagaagttgttgatgagttcatggagaatgttccaatgtcggttagactcgcaaagtttcgaaccaaatcctcaaaaagaggtccgaaaaataataataatttaggtaaggggcgttcaggcggaaggcctaaaccaaaaagttttgatgaagttgaaaaagagtttgataatttgattgaagatgaagccgagacgtcggtcgcagattctgattcgtattaattaa

SEQ ID NO: 35 - DNA, 51 bp, Artificial Sequence
  primer
gtatatgcggccgcgtgtgagtgcatgaaaaaaggcgttatgcgacccggc

SEQ ID NO: 36 - DNA, 51 bp, Artificial Sequence
  primer
tggatagaattctggccaaagagatcaggtcagcacggtcatgaatcgttc

SEQ ID NO: 37 - DNA, 51 bp, Artificial Sequence
  primer
gtatatgcggccgcgtgtgagtgcatgaacaaaggtgtaatgcgccccggc

SEQ ID NO: 38 - DNA, 51 bp, Artificial Sequence
  primer
tggatagaattctggccaaagagattaggtcataacggtcatgaatcgttc

SEQ ID NO: 39 - DNA, 27 bp, Artificial Sequence
  primer
gtatatgcggccgcgtgtgagtgcatg

SEQ ID NO: 40 - DNA, 27 bp, Artificial Sequence
  primer
tggatagaattctggccaaagagatta